(19) United States
(12) Patent Application Publication
(10) Pub. No.: US 2026/0273177 A1
(43) Pub. Date: Sep. 17, 2026

(54) FLUID DELIVERY CASSETTE

(71) Applicant: Eli Lilly and Company, Indianapolis, IN (US)

(72) Inventors: Jared Alden JUDSON, Medford, MA (US); Anthony Lawrence SCHAFF, Carmel, IN (US)

(21) Appl. No.: 18/873,853

(22) PCT Filed: Jun. 20, 2023
(86) PCT No.: PCT/US2023/025704

§ 371 Date: Dec. 11, 2024

Related U.S. Application Data
(60) Provisional application No. 63/354,323, filed on Jun. 22, 2022.

(51) Int. Cl. A61M 5/145

(52) U.S. Cl. CPC A61M 5/14566; A61M 2005/14573

(57) ABSTRACT

A fluid delivery cassette includes a cartridge assembly and a needle assembly. The cartridge assembly includes a cartridge configured to contain a fluid, a septum positioned at a distal opening of the cartridge, a piston sealing a proximal opening of the cartridge and movable in an axial direction, and a drive assembly configured to move the piston. The drive assembly includes an axially extendible portion having a distal end that is controllably extendible from a retracted position to an extended position, and a rotatable force-transfer portion configured to receive an applied force and to cause the distal end of the axially extendible portion to move in the axial direction in response to the applied force. The axially extendible portion can be manufactured as a tapered helix having a diameter that varies so that the axially extendible portion is collapsible into the retracted position.

### FIELD OF THE DISCLOSURE

The present disclosure relates generally to fluid delivery devices, such as injection devices. More specifically, the present disclosure relates to a fluid delivery cassette including a retractable needle and a drive assembly for delivering a controlled amount of fluid through the needle.

### BACKGROUND OF THE DISCLOSURE

Conventional injection devices are often used to inject a medication into a patient via a needle. It is sometimes advantageous for the medication to be administered without the presence of a medical professional, such as when the medication is to be administered frequently (e.g., daily at different times during each day). However, it may be a challenge to ensure that needles are maintained in a sterile environment prior to use in injections, as well to ensure that the medication is administered safely, efficiently, and in a proper amount. Furthermore, some patients are uncomfortable with seeing or directly handling needles.

An injection pen is an example of a conventional injection device. Typical injection pens generally include a rigid rod that acts on a piston within a cartridge. As the rod advances the piston during an injection process, the medication in the cartridge is dispensed through a needle into the patient. In order to accommodate the rod, conventional injection pens typically are long and thin, with a length of the injection pens typically being more than twice the length of a cartridge barrel in which the medication is contained. Similarly, for conventional non-pen-shaped refillable injection devices, a length of the devices is typically more than twice the length of a cartridge barrel in which the medication is contained.

When conventional injection devices are used to self-administer medication frequently, it may be desirable for the injection devices to be convenient to use and easily carried by users. For example, as noted above, diabetes patients often self-administer insulin using injection devices and carry the devices with them throughout the day. Although conventional injection pens and similar conventional devices are sufficiently small to be portable, such devices have lengths that often make transport of the devices awkward.

### SUMMARY

According to an aspect of the present technology, a fluid delivery cassette may comprise a cartridge assembly and a needle assembly attached to the cartridge assembly. The cartridge assembly may comprise a cartridge configured to contain a fluid; a septum positioned at a distal opening of the cartridge; a movable piston sealing a proximal opening of the cartridge, the piston being movable in an axial direction of the cartridge; and a drive assembly configured to move the piston in the axial direction toward the distal opening. The drive assembly may comprise an axially extendible portion having a distal end that is controllably extendible from a retracted position to at least one extended position along a longitudinal axis and a rotatable force-transfer portion configured to receive an applied force and to cause the distal end of the axially extendible portion to move in the axial direction in response to the applied force. The axially extendible portion may be manufactured as a tapered helix having a diameter that varies from a first end of the tapered helix to a second end of the tapered helix so that the axially extendible portion is collapsible into the retracted position.

According to another aspect of the present technology, a fluid delivery system may comprise a disposable dispenser; a reusable cassette housing; and a reusable motor assembly. The dispenser may comprise a cartridge assembly and a needle assembly attached to the cartridge assembly. The cassette housing may be configured to contain at least a portion of the dispenser. The motor assembly may be configured to drive movement in the cartridge assembly or in the needle assembly or in both the cartridge assembly and the needle assembly, and may be removably mountable on the cassette housing, The cartridge assembly may comprise a cartridge configured to contain a fluid; a movable piston sealing a proximal opening of the cartridge, the piston being movable in an axial direction of the cartridge; and a drive assembly configured to move the piston in the axial direction toward a distal end of the cartridge. The drive assembly may comprise an axially extendible portion having a distal end that is controllably extendible from a retracted position to at least one extended position; and a rotatable portion configured to be driven by the motor assembly to cause the distal end of the axially extendible portion to move in the axial direction. The axially extendible portion may be manufactured as a tapered helix having a diameter that varies from a first end of the tapered helix to a second end of the tapered helix so that the axially extendible portion is collapsible into the retracted position.

According to a further aspect of the present technology, a method of using a fluid delivery cassette may comprise activating a needle assembly of a dispenser by fully inserting the dispenser in a cassette housing and causing a latch mechanism of the cassette housing to latch to the dispenser to prevent movement of the needle assembly relative to the cassette housing; rotating a needle selector of the needle assembly to select a needle of the needle assembly; and causing a cartridge assembly of the dispenser to eject a fluid in a cartridge of the cartridge assembly through the needle of the needle assembly by: causing an axially extendible portion to extend from a retracted position to an extended position by forming an elongating helix, and using the elongating helix to apply pressure on a piston in the cartridge to eject the fluid from the cartridge into the needle. The needle assembly may be prevented from being activated when the dispenser is not fully inserted in the cassette housing.

According to another aspect of the present technology, a method of assembling a fluid delivery cassette may comprise method comprising providing a ribbon formed of a resilient material and defining a tapered helix having a diameter that varies from a first end of the tapered helix to a second end of the tapered helix, and applying an axial force against an end of the ribbon in the tapered helix to axially collapse the ribbon, thereby forming a coil. The axial force may be a force along an axis of the tapered helix. The ribbon in the tapered helix may include edges that are uncoupled to one another. The method also may comprise inserting the coil inside a ribbon chamber of a cartridge housing. The cartridge housing may include a cartridge configured to contain a fluid, a septum positioned at a distal opening of the cartridge, and a movable piston sealing a proximal opening of the cartridge. Movement of the ribbon along the cartridge housing may cause an extended portion of the ribbon to form a columnar helix when the edges of the ribbon are coupled to one another. The columnar helix may have a consistent diameter sized to fit within the cartridge in order to movably engage with the piston.

It is noted that fluid delivery cassettes having various different features are disclosed herein and these features may be combined in various different configurations, including configurations not specifically illustrated or discussed. Although several different combinations of such features are described herein, a person having ordinary skill in the art will realize that further such combinations not explicitly described herein are also possible and enabled by the present disclosure and are within the scope of the present application. Additionally, although various techniques are disclosed herein for attaining the disclosed features, a person having ordinary skill in the art will realize that some modifications to the disclosed techniques may be possible and within the scope of the disclosed techniques. It is also to be understood that the phraseology and terminology employed herein are for the purpose of description and should not be regarded as limiting.

### BRIEF DESCRIPTION OF THE DRAWINGS

Various aspects, techniques, and embodiments of the present technology disclosed herein are described below with reference to the accompanying drawings. It should be appreciated that the figures are not necessarily drawn to scale. Items appearing in multiple figures may be indicated by the same reference numeral. For purposes of clarity, not every component may be labeled in every figure. Features of the present technology will become more apparent, and techniques for how to attain the features of the present technology, will be better understood by reference to the following detailed description considered in conjunction with the accompanying drawings, wherein:

FIG. 1 shows a perspective view of a dispenser comprising a cartridge assembly and a needle assembly.

FIG. 2 shows a perspective view of the cartridge assembly of FIG. 1.

FIG. 3 shows a perspective view of the cartridge assembly of FIG. 1 in a partially disassembled state.

FIG. 4A shows a perspective view of a longitudinal cross-section of the cartridge assembly of FIG. 1.

FIG. 4B shows another perspective view of a longitudinal cross-section of the cartridge assembly of FIG. 1.

FIG. 4C shows a perspective view of cross-section of the cartridge assembly of FIG. 1 cut along a plane 4C in FIG. 4A.

FIG. 4D shows a plan view of a slice of the cartridge assembly of FIG. 1 cut along parallel planes 4D in FIG. 4A.

FIG. 4E shows a plan view of a proximal end of the cartridge assembly of FIG. 1.

FIG. 5 schematically shows an plan view of a longitudinal cross-section of a portion of a proximal end of a first variation 5A and a second variation 5B of the cartridge assembly of FIG. 1.

FIGS. 6A and 6B show elevational side views of an axially extendible ribbon. In FIGS. 6A and 6B, a distal end of the ribbon is on the left and a proximal end of the ribbon is on the right. In FIGS. 6A and 6B, the ribbon is in an unstressed state and has a configuration of a tapered helix.

FIG. 6C shows an elevational end view of the axially extendible ribbon of FIG. 6A in the unstressed state. In FIG. 6C, an external surface of the distal end of the ribbon is shown.

FIG. 6D shows an elevational end view of the axially extendible ribbon of FIG. 6A in the unstressed state. In FIG. 6D, an internal surface of the distal end of the ribbon is shown.

FIG. 7A shows a perspective view of a cartridge assembly and a driver gear configured to rotationally drive a portion of the cartridge assembly.

FIG. 7B shows another perspective view of the cartridge assembly and the driver gear of FIG. 7A.

FIG. 7C shows a perspective view of the driver gear of FIG. 7A.

FIG. 8 shows a perspective view of a dispenser comprising a cartridge assembly and a needle assembly.

FIG. 9A shows a perspective view of the needle assembly of FIG. 8.

FIG. 9B shows a plan view of a cross-section of the needle assembly of FIG. 9A cut along a plane 9B in FIG. 9A. In FIG. 9B, a surface on the left of the plane 9B is shown.

FIG. 9C shows a perspective view of the needle assembly of FIG. 9A in a partially disassembled state.

FIG. 10 shows a perspective view of a cassette comprising a dispenser partially inserted in a cassette housing. In FIG. 10, driver gears for driving movement in the dispenser are shown.

FIGS. 11A and 11B show a perspective view of a partial longitudinal cross-section of a needle assembly partially inserted and fully inserted, respectively, in a cassette housing. In

FIG. 11A, a gear lock of the needle assembly is in a locked state. In FIG. 11B, the gear lock is in an unlocked state.

FIG. 12A shows a perspective view of a distal section of a dispenser.

FIG. 12B shows the same view as FIG. 12A but with a housing of a needle assembly of the dispenser appearing translucent.

FIG. 12C shows a perspective view of the distal section of the dispenser of FIG. 12B in a partially disassembled state.

FIGS. 13A and 13B show plan views of a longitudinal cross-section of a needle assembly in a locked state and an unlocked state, respectively. In FIG. 13A, a transfer gear (idler) is prevented from rotating a needle-movement gear of the needle assembly by a spring pushing a lock plate against a housing protrusion. In FIG. 13B, the transfer gear (idler) and the needle-movement gear are in an engaged state, with the transfer gear (idler) being spaced apart from the housing protrusion and therefore not prevented from rotating the needle-movement gear.

FIG. 13C shows a plan view of a lateral cross-section of the needle assembly of FIG. 13A cut through the lock plate, showing portions of the lock plate blocking rotational movement of a needle selector.

FIG. 13D shows a plan view of a lateral cross-section of the needle assembly of FIG. 13A cut through the transfer gear (idler), showing lips comprising the housing protrusion blocking rotational movement of the transfer gear.

FIG. 14A shows an elevational side view of a distal section of a cassette.

FIG. 14B shows a perspective view of the distal end of the cassette of FIG. 14A. In FIG. 14B, a latch of a housing of the cassette is latched to a dispenser of the cassette, preventing the dispenser from moving relative to the housing.

FIG. 14C shows a perspective view of the distal end of FIG. 14B. In FIG. 14C, the latch is unlatched from the dispenser, enabling a biased spring of a gear lock of the dispenser to move the dispenser outwards relative to the housing of the cassette.

FIG. 14D shows a perspective view of the distal end of FIG. 14C. In FIG. 14D, after being unlatched, the dispenser is movable out of the housing of the cassette and a new dispenser is movable into the housing the cassette.

FIG. 15A shows a plan view of a longitudinal cross-section of a distal end of a cassette. In FIG. 15A, a latch of a housing of the cassette is latched to a latch receiver of a dispenser of the cassette, preventing the dispenser from moving relative to the housing.

FIG. 15B shows a plan view of a longitudinal cross-section of the distal end of FIG. 15A. In FIG. 15B, the latch of the housing of the cassette is unlatched from the latch receiver of the dispenser, enabling a biased spring of a gear lock of the dispenser to move the dispenser outwards relative to the housing.

FIG. 15C shows a plan view of a longitudinal cross-section of the distal end of FIG. 15B. In FIG. 15C, after being unlatched, the dispenser is movable out of the housing of the cassette and a new dispenser is movable into the housing.

FIG. 16A shows an elevational side view of a distal section of a cassette.

FIG. 16B shows a perspective view of the distal end of the cassette of FIG. 16A. In FIG. 16B, a latch-release actuator of the cassette is in a hold position preventing a dispenser from moving relative to a housing of the cassette.

FIG. 16C shows a perspective view of the distal end of FIG. 16B. In FIG. 16C, the latch-release actuator is moved to a release position by a lateral force, which enables a latch to pivot to disengage from an engagement surface of the dispenser, and which in turn enables a biased spring of a gear lock of the dispenser to move the dispenser outwards relative to the housing.

FIG. 16D shows a perspective view of the distal end of FIG. 16C. In FIG. 14D, after being disengaged, the dispenser is movable out of the housing of the cassette and a new dispenser is movable into the housing the cassette.

FIG. 17A shows a plan view of a longitudinal cross-section of a distal end of a cassette. In FIG. 17A, a latch of a housing of the cassette is latched to an engagement surface protruding from a dispenser of the cassette, preventing the dispenser from moving relative to the housing.

FIG. 17B shows a plan view of a longitudinal cross-section of the distal end of FIG. 15A. In FIG. 17B, the latch of the housing of the cassette is pivoted by an actuator to disengage from the engagement surface protruding from the dispenser, enabling a biased spring of a gear lock of the dispenser to move the dispenser relative to the housing of the cassette.

FIG. 17C shows a plan view of a longitudinal cross-section of the distal end of FIG. 17B. In FIG. 17C, after being disengaged, the dispenser is movable out of the housing of the cassette and a new dispenser is movable into the housing.

FIGS. 18A and 18B show, respectively, a perspective view of a cassette housing during insertion of a dispenser into the cassette housing and a perspective view of the dispenser fully inserted in the cassette housing. In FIGS. 18A and 18B, a driver gear for rotating a needle-selection gear of the dispenser is positioned at an opening through which the driver gear rotates the needle-selectin gear.

FIG. 18C shows an enlarged perspective view of a compartment of the cassette housing of FIG. 18B. In FIG. 18C, a needle assembly of the dispenser is fully inserted the compartment and the driver gear is not shown at the opening, such that a portion of the needle-selection gear is visible through the opening. Although not fully visible in FIG. 18C, a portion of another opening is shown, through which another driver gear may rotate a needle-movement gear of the dispenser (e.g., via a transfer gear).

FIG. 19 shows a block diagram of a motor assembly comprising a plurality of driver assemblies and a controller for controlling the driver assemblies.

FIG. 20A shows a perspective view of a cassette comprising a dispenser fully inserted in a housing of the cassette. In FIG. 20A, a driver gear is engaged with a drive assembly of the dispenser, and a cartridge of the dispenser is aligned with and visible through a window of the housing.

FIG. 20B shows a perspective view of the cassette of FIG. 20A while the dispenser is being inserted in the housing.

FIG. 21 shows a block diagram of a medication delivery device including a cassette, a motor assembly, and a controller.

### DETAILED DESCRIPTION

Provided herein are examples of fluid delivery cassettes, as well as examples of components included in the fluid delivery cassettes, examples of systems that can use the fluid delivery cassettes, and techniques for attaining the fluid delivery cassettes. As will be appreciated, although some examples of the fluid delivery cassettes are described herein in connection with administering a medication to a patient, such cassettes are not limited to use in medical applications and may additionally or alternatively be used in other non-medical applications (e.g., where a precise amount of fluid is to be delivered).

The inventors have recognized and appreciated that various factors can be important for fluid delivery cassettes. In particular, while ease of use of a fluid delivery cassette can be important, minimizing medical waste may also be important. The fluid delivery cassettes provided herein may enable multiple needles to be provided and individually activated to deliver multiple doses of medication from a single vial or fluid chamber, thus reducing waste associated with multiple needle packages and/or multiple single-dose vials of medication. The multiple doses may be injected at different times using different ones of the needles or the doses may be injected at different times using a single one of the needles. For example, a patient may use one needle for multiple injections for one day, and may use another needle for multiple injections for another day. In some embodiments, single needle configurations can also be used according to the techniques described herein.

Various embodiments of the fluid delivery cassettes provided herein may enable precise amounts of fluid to be injected from a single vial or fluid chamber through controlled movement of a piston. For example, by controlling a precise amount of rotation of a driver gear (e.g., via a computer-controlled motor), the driver gear may cause a length of an extendible ribbon to expand to push the piston by a precise amount. One of the advantages of the fluid delivery cassettes provided herein may be that a configuration of the extendible ribbon and a drive assembly for driving expansion and retraction of the ribbon may enable the cassettes to be compact and have a relatively short length. Such compact cassettes can still provide accurate fluid control by using various techniques described herein.

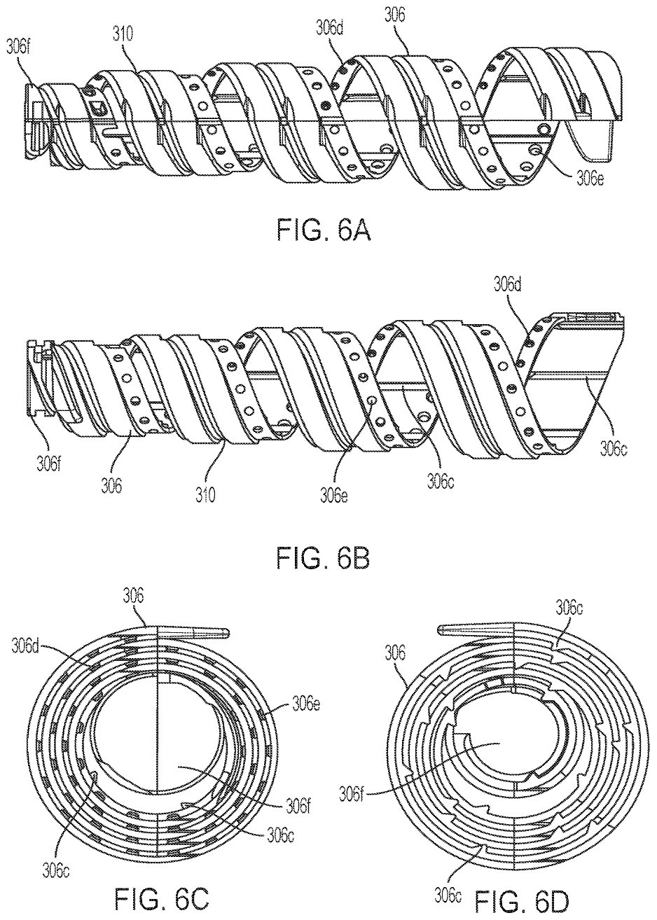

Conventionally, the ribbon is manufactured in a tubular shape, such that the ribbon forms a helix that is cylindrical or tubular, and such that a diameter from a first end of the helix does not substantially vary to a second end of the helix. The inventors have recognized and appreciated that such a tubular shape requires a radial force to be exerted to wind the helix into a coil and, in addition, requires an axial force to compress or flatten the helix into a coil. Accordingly, such a tubular shape can be time consuming and/or difficult to work with when rearranging the helix into a coil for installation into a fluid delivery cassette or into a component of a fluid delivery cassette, such as a ribbon chamber. The inventors therefore developed techniques that provide for a tapered helix. When not confined in a ribbon chamber and in an absence of a force exerted on the ribbon (e.g., in an unstressed state), the ribbon may form a tapered helix having a diameter that varies from a first end of the tapered helix to a second end of the tapered helix (e.g., as depicted in FIGS. 6A and 6B). The tapering may be formed along the cross-sectional area of the helix, increasing along a longitudinal axis (e.g., axis A in FIG. 2) from the first end to the second end. The tapered helix may or may not have a gap between segments or loops of the helix when seen in a side view. Such a tapered structure may facilitate manufacturing of a cartridge assembly, as discussed herein. For example, with a tapered structure, the ribbon may be easily collapsed into a coil by applying an axial force to squeeze the first and second ends toward each other (e.g., by a pinching action between two fingers), and may facilitate insertion of the coil onto a spindle and into a ribbon chamber, as discussed herein. As a result, the tapered helix can be pressed into a coil without needing to also exert a radial force to wind the helix around itself.

The fluid delivery cassettes may include reusable portions and disposable portions. In various embodiments of the cassettes disclosed herein, a dispenser with one or more needles and/or fluid to be injected by the one or more needles may be disposable. In various embodiments of the cassettes disclosed herein, a housing configured to accommodate the dispenser and/or to support one or more driver assemblies for driving movement in the dispenser may be reusable. The one or more driver assemblies and a controller for controlling the one or more driver assemblies may also be reusable. In some embodiments, a fluid delivery cassette according to the present technology may be part of a medication delivery device. For example, a housing of the medication delivery device may house the fluid delivery cassette, a motor assembly configured to drive movement in the fluid delivery cassette, and a controller (e.g., a computer processor) configured to control the motor assembly.

Following below are more detailed descriptions of various concepts related to, and embodiments of, techniques described above. It should be appreciated that various aspects described herein may be implemented in any of numerous ways. Examples of specific implementations are provided herein for illustrative purposes only. In addition, the various aspects described in the embodiments below may be used alone or in any combination, and are not limited to the combinations explicitly described herein.

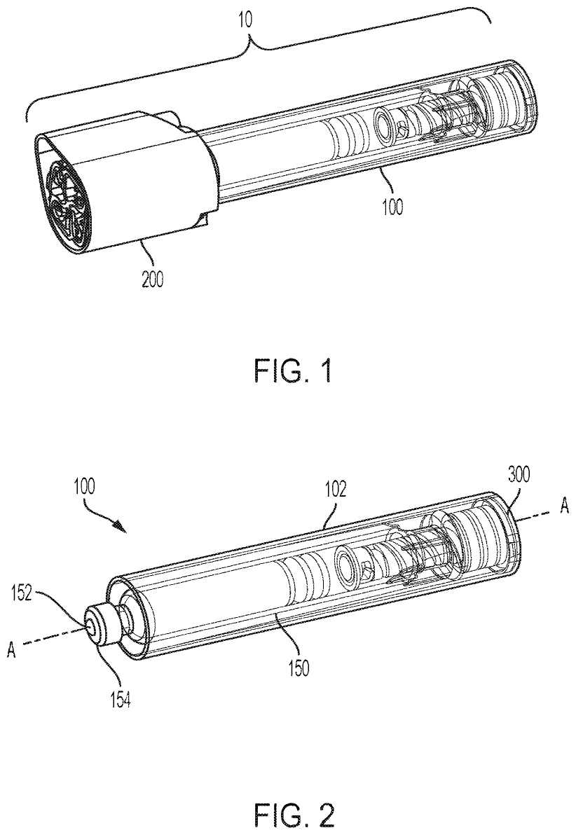

Turning now to the figures, FIG. 1 shows a perspective view of a dispenser 10 comprising a cartridge assembly 100 and a needle assembly 200, according to some embodiments of the present technology. The dispenser 10 may be used in a fluid delivery cassette, as discussed below. The needle assembly 200 may be located at a distal end of the dispenser 10. For the sake of clarity, the term “distal” may be used herein to identify a location closer to an outlet of a needle of the needle assembly (e.g., closer to an end that comes into contact with a patient to be injected with the needle), and the term “proximal” may be used herein to identify a location farther away from the outlet of the needle. As discussed below, the dispenser 10 may form an aspect of a medication delivery device. While some aspects of the medication delivery device may be described herein and/or shown in the drawings, it should be appreciated that some aspects of the medication delivery device may not be shown in the figures and/or described, or may not be shown in the figures and/or described in detail. For example, a housing of the medication delivery device may house the dispenser 10. The housing of the medication delivery device may include other components and electronics to make the dispenser operable. For example, the housing may also include one or more motors for driving movement in or relative to the dispenser 10, and a computer processor for controlling operation of the one or more motors.

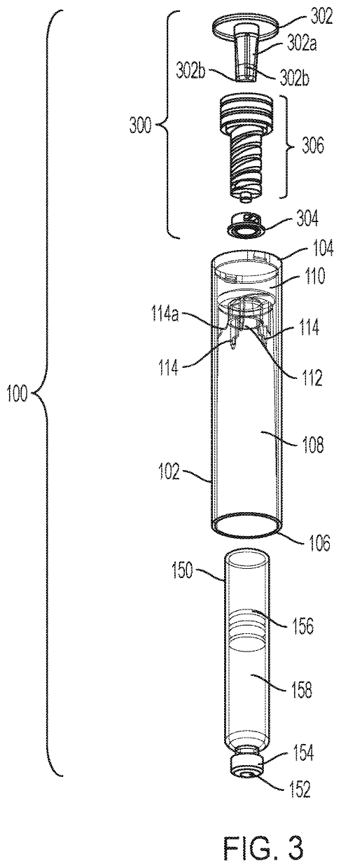

FIG. 2 shows a perspective view of the cartridge assembly 100 in an assembled state, and FIG. 3 shows a perspective view of the cartridge assembly 100 in a partially disassembled state, according to some embodiments of the present technology. The cartridge assembly 100 may comprise a cartridge 150 configured to hold a fluid (e.g., a liquid medication) therein. The fluid may be confined to a fluid chamber 158 delimited by a piston 156 configured to seal a proximal opening of the cartridge 150, and delimited by a septum 152 configured to seal a distal opening of the cartridge 150. A septum retainer 154 may be coupled to a distal end of the cartridge 150 to hold the septum 152 in place at the distal opening of the cartridge 150. In some embodiments, the piston 156 may be configured to provide a movable seal against an internal surface of the cartridge 150, to prevent the fluid in the fluid chamber 158 from leaking out of a proximal end of the fluid chamber 158 even during movement of the piston 156 along the internal surface of the cartridge 150. The septum 152 may be formed of a self-sealing material (e.g., an FDA-rated elastomeric material) that may be pierced by a piercing object of the needle assembly 200 during an injection process, to enable the fluid in the fluid chamber 158 to flow out of the fluid chamber 158 during the injection process, and that may reseal to provide a fluid-tight seal after the piercing object is retracted from the septum 152. In some embodiments, the retraction of the piercing object out of the septum 152 and the subsequent resealing of the septum 152 may prevent contamination of the fluid remaining in the fluid chamber 158, such that the remaining fluid may be used in future injection processes. In some embodiments, the fluid in the fluid chamber 158 may be sufficient for multiple injections (e.g., multiple doses of medication).

Devices according to the present disclosure may carry and dispense one or more liquid medications, which may also be referred to as medications or drugs and maybe held in the fluid chamber 158. Such medications may include, for example, epinephrine, anaesthetics, analgesics, steroids, insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, combined GIP/GLP-1 agonists such as tirzepatide, basal insulins, oxyntomodulin analogs, oxyntomodulin derivatives, therapeutic antibodies including but not limited to IL-23 antibody analogs or derivatives, such as mirikizumab, IL-17 antibody analogs or derivatives, such as ixekizumab, therapeutic agents for pain-related treatments, such as galcanzeumab or lasmiditan, or lebrikizumab and any therapeutic agent that is capable of delivery by the devices described herein. Devices according to the present disclosure may be operated in a manner generally as described herein by a user (for example, a healthcare professional, a caregiver, or another person) to deliver one or more medications to a patient (for example, another person or the user).

The cartridge 150 may be held in a housing 102 of the cartridge assembly 100. In some embodiments of the present technology, the housing 102 may have a cylindrical shape, as depicted in FIG. 3, and a plurality of ribs 114 may extend radially from an internal surface of the housing 102. The ribs 114 may serve as spacers that keep the cartridge 150 at a predetermined position in a cartridge chamber 108 of the housing 102. In some embodiments, the ribs 114 may have rib shoulders 114a configured to serve as an axial insertion limit of the cartridge 150 in the housing 102. In some embodiments, when the cartridge 150 is fully inserted in the cartridge chamber 108, an edge at a proximal end of the cartridge 150 may abut the rib shoulders 114a and the cartridge 150 may be radially centered in the cartridge chamber 108. In some embodiments, when the cartridge 150 is fully inserted in the cartridge chamber 108, the septum 152 may extend out of a distal end of the housing 102, such that the septum 152 may be positioned in a recess of the needle assembly 200.

In some embodiments of the present technology, the cartridge assembly 100 may comprise a drive assembly 300 configured to drive movement of the piston 156 to eject the fluid in the fluid chamber 158 out of the distal end of the cartridge 150. The drive assembly 300 may be referred to as a “driven” assembly because the drive assembly 300 may itself be driven to move and, in turn, may convert a received drive force to a force that causes the piston 156 to move along an axis A of the cartridge assembly 100. In some embodiments, the drive assembly 300 may comprise a driven mechanism 302, which may be a drive gear 302 configured to be driven to rotate about the axis A. The driven mechanism 302 may comprise a spindle 302a around which an axially extendible member 306 may be wound. In some embodiments, the axially extendible member 306 may be a ribbon (element 306 may refer to either the axially extendible member or the ribbon interchangeably) having a distal end attached to a foot 304. The foot 304 may be configured to rotate relative to the distal end of the rotating ribbon 306. The drive assembly 300 may be configured such that rotation of the driven mechanism 302 causes the ribbon 306 to extend or expand along the axis A when the driven mechanism 302 is rotated in a first direction. In some embodiments, the driven mechanism 302 may cause the ribbon 306 to retract when the driven mechanism 302 is rotated in a second direction opposite to the first direction. As discussed below, when the ribbon 306 is driven to extend, an axial distance between the distal and proximal ends of the ribbon 306 expands or increases, causing the foot 304 to be moved axially. When the foot is in contact with the piston 156, expansion of the ribbon 306 causes the piston 156 to move axially toward the distal opening of the cartridge 150. In some embodiments, precise control of an amount of rotation of the driven mechanism 302 may cause a precise amount of expansion of the ribbon 306 and a precise amount of movement of the piston 156, which in turn may cause a precise amount of fluid to be ejected.

Additional ribbon-expansion mechanisms are described in WO2017/165154A1 and WO2019/112866A1, both of which are incorporated herein in their entireties.

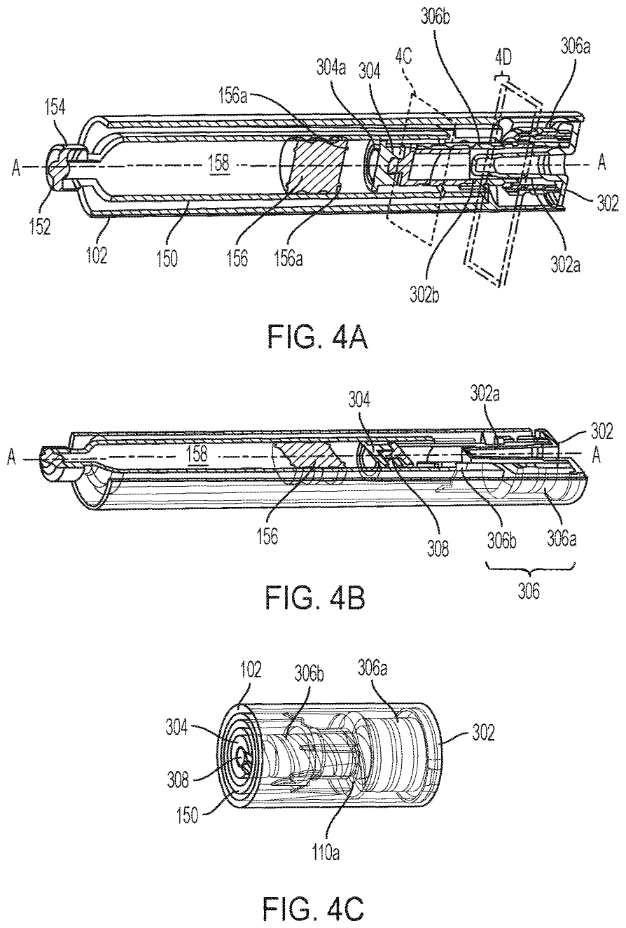

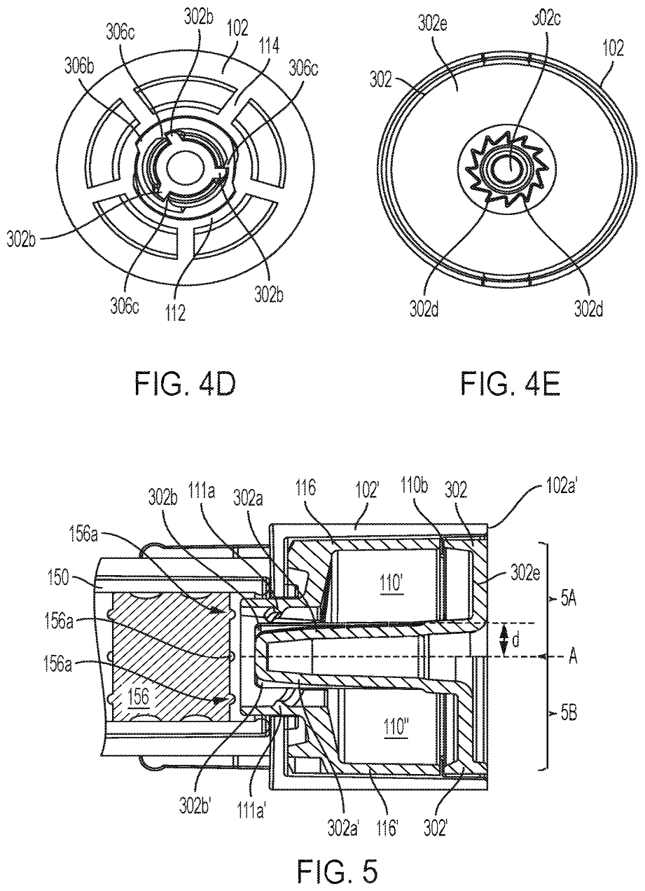

FIGS. 4A and 4B show perspective views of longitudinal cross-sections of the cartridge assembly 100, according to some embodiments of the present technology. FIG. 4C shows a perspective view of cross-section of the cartridge assembly 100 cut along a plane 4C in the view of the cartridge assembly 100 shown in FIG. 4A, according to some embodiments of the present technology. In FIGS. 4A and 4B, the foot 304 of the drive assembly 300 is spaced apart from the piston 156. As will be appreciated, during an injection process, the foot 304 may be in contact with the piston 156. A distal surface of the foot 304 may comprise a groove 304a configured to engage with protrusions 156a on a proximal surface of the piston 156. For example, the groove 304a may be circular and the protrusions 156a may be bumps configured to fit in the groove 304a when the foot 304 and the piston 156 are abutted to each other. In some embodiments, the foot 304 may be configured to rotate about the axis A while the protrusions 156a remain rotationally fixed when the piston 156 is being pushed by the foot 304.

FIG. 4D shows a plan view of a slice of the cartridge assembly 100 cut along parallel planes 4D in the view of the cartridge assembly 100 shown in FIG. 4A, according to some embodiments of the present technology. In some embodiments, the foot 304 may be connected to the distal end of the ribbon 306 by a bearing 308, which may rotate during expansion and retraction of the ribbon 306 and which may enable the foot 304 to remain rotationally stationary during expansion and retraction of the ribbon 306. The proximal surface of the foot 304 may have a socket recess configured to receive a ball-shaped protrusion of the bearing 308, which may rotate in the socket recess during expansion and retraction of the ribbon 306.

According to some embodiments of the present technology, the ribbon 306 may be wound around the spindle 302a such that, when the ribbon 306 is in a fully retracted state, some or all of the ribbon 306 may be coiled, such that loops of the coil may have proximal edges that are generally aligned with each other and distal edges that are generally aligned with each other. Thus, when the ribbon 306 is fully retracted, the coil may be compact and may fit in a ribbon chamber 110 of the housing 102 located at a proximal end 104 of the housing 102. In some embodiments, when the driven mechanism 302 is rotated to expand the ribbon 306, axially elongated spindle ribs 302b protruding radially from a longitudinal portion of the spindle 302a may bear against rib latches 306c protruding from a surface of the ribbon 306, causing the ribbon 306 to rotate and uncoil. In some embodiments, the spindle ribs 302b and the rib latches 306c may be structured to have complementary angles to latch or hook to each other, as depicted in FIG. 4D. In some embodiments, the ribbon 306 may uncoil to form a helix having an axial length controlled by an amount of rotation of the driven mechanism 302. Thus, through controlled rotation of the driven mechanism, controlled movement of the piston 156 may be achieved and, consequently, controlled ejection of the fluid in the fluid chamber 158 may be achieved. In some embodiments, the cartridge assembly 100 may be structured such that a predetermined number of rotations of the driven mechanism 302 may result in movement of the foot 304 by 0.2 mm or 0.3 mm or 0.4 mm or 0.5 mm. In some embodiments, one complete rotation (e.g., a rotation of 360°) may result in a known amount of movement of the foot 304 and/or a known amount of movement of the piston 156 pushed by the foot 304.

According to some embodiments of the present technology, the ribbon 306 may be uncoiled from “inside to outside” such that an innermost loop of the coil in contact with the spindle ribs 302b uncoils first and extends or expands to form a growing helix. For example, FIGS. 3, 4A, and 4B depict a retracted portion 306a of the ribbon 306 formed of outer coil loops of the ribbon 306, and depict an extended portion 306b of the ribbon 306 extending from an inner coil loop of the ribbon 306 to form a helix.

The helix formed from an extended portion 306b of the ribbon 306 may be guided from the ribbon chamber 110 of the housing 102 to the cartridge chamber 108 of the housing by a helix guide 112. An unextended or retracted portion 306a of the ribbon 306 may remain coiled in the ribbon chamber 110. In some embodiments of the present technology, the helix guide 112 may be a tubular section of the housing 102 having a diameter that is smaller than a diameter of the cartridge chamber 108. A diameter of the helix may correspond to the diameter of the helix guide 112. In some embodiments, the ribs 114 of the housing 102 may extend radially from the internal surface of the housing 102 and may contact an external surface of the helix guide 112, as depicted in FIG. 4D.

FIG. 4E shows a plan view of a proximal end 104 of the cartridge assembly 100, according to some embodiments of the present technology. The driven mechanism 302 may fit in the ribbon chamber 110 of the housing 102 and may rest on a lip 110b extending from an inner surface of the ribbon chamber 110 (see FIG. 5). The driven mechanism 302 may be structured to engage with and be rotated by a driver, discussed below. In some embodiments, a surface of the driven mechanism 302 may comprise a recess 302c configured to receive a spline shaft therein. The recess 302c may be formed in a spindle base 302e of the spindle 302a from which the longitudinal portion extends therefrom. The spindle base 302e may include a disk-shaped body having a spindle-base diameter. The recess 302c may have recessed teeth 302d configured to mate with corresponding teeth of the spline shaft. As will be appreciated, the driven mechanism 302 may be configured with other structures that enable the driven mechanism 302 to be rotated. For example, instead of the recess 302c, the surface of the driven mechanism 302 may be provided with a shaft (e.g., a spline shaft) configured to engage with and be rotated by a motorized apparatus having a recess (e.g., a spline-shaped recess) that mates with the shaft.

FIG. 5 schematically shows an plan view of a longitudinal cross-section of a portion of a proximal end of a first variation 5A and a second variation 5B of the cartridge assembly 100, according to some embodiments of the present technology. The first and second variations 5A, 5B may be comprised a housing 102′ that is a variation of the housing 102. The first variation 5A may comprise a ribbon container 116 that may be insertable in the housing 102, which may facilitate manufacturing by enabling a ribbon bobbin comprised of the ribbon container 116, the ribbon 306, and the driven mechanism 302 to be manufactured separately and to be easily installed as a unit in the housing 102′. In some embodiments, a ribbon chamber 110′ of the ribbon container 116 may have a distal base that is sloped to facilitate helix formation by providing room for inner loops of the coil to uncoil while outer loops are relatively more confined to stay together in the coil. The second variation 5B may comprise a ribbon container 116′ that is similar to the ribbon container 116 but may have a ribbon chamber 110″ that is smaller in volume than the ribbon chamber 110′ of the first variation 5A, with an axially shorter dimension resulting in an axially tighter ribbon chamber 110″. The axially tighter ribbon chamber 110″ may be sized closely to associate with an edge-to-edge width of the ribbon in order to facilitate a reduction of binding and slippage when the ribbon 306 is moving to an extended configuration. The second variation 5B may comprise a driven mechanism 302′ that has a recessed outer surface, in which a peripheral edge of the driven mechanism 302′ may be flush with the proximal edge 102a′ of the housing 102′ but a remainder of the driven mechanism 302′ may be sunken relative to the proximate edge 102a′. Such a sunken configuration may advantageously enable a more secure engagement of the driven mechanism 302′ with a driver, thus enabling a higher degree of confidence that a rotation or a partial rotation of the driven mechanism 302′ will result in a predictable axial movement of the plunger 156. In another variation, the ribbon container 116, 116′ may not be a component that is insertable in the housing 102′ but may instead be an integral structure of the housing 102′. For example, the housing 102′ may formed of molded plastic such that a ribbon chamber of the housing 102′ may have an internal structure corresponding to the ribbon container 116, 116′. As depicted in FIG. 5, a surface of the spindle 302a, 302a′ may have a tapered profile, such that a diameter of the spindle 302a, 302a′ may decrease gradually from a relatively larger diameter at a proximal end of the spindle 302a, 302a′ to a relatively small diameter at a distal end of the spindle 302a, 302a40 . In some embodiments, the spindle 302a, 302a′ may have a conical shape. The tapered profile of the spindle 302a, 302a′ may enable a smooth movement of the ribbon 306 relative to the spindle 302a, 302a′ as the helix axially expands in length. For example, the tapered profile may enable an innermost coil of the ribbon 306 to uncoil gradually and become part of the expanding helix. In some embodiments, the spindle ribs 302b, 302b′ may extend radially from the tapered surface of the spindle 302a, 302a′ such that a radial distance d of a radially outer edge of each of the spindle ribs 302b, 302b′ to the axis A may be the same along an axial length of each of the spindle ribs 302b, 302b′, as depicted in FIG. 5. Such a configuration of spindle ribs 302b, 302b′ may cause the helix to have a columnar shape.

FIGS. 6A and 6B show elevational side views of the ribbon 306 in an unstressed state, according to some embodiments of the present technology. In FIGS. 6A and 6B, a distal end of the ribbon 306 is on the left and a proximal end of the ribbon 306 is on the right. The distal end of the ribbon may be structured as a latch 306f that may engage directly with the foot 304 or that may engage with the bearing 308, discussed herein.

According to some embodiments of the present technology, the ribbon 306 may be formed of a resilient material such that, when not confined in the ribbon chamber 110 and in an absence of a force exerted on the ribbon 306 (e.g., in the unstressed state), the ribbon 306 may form a tapered helix having a diameter that varies from a first end of the tapered helix to a second end of the tapered helix, as depicted in FIGS. 6A and 6B. The tapering may be formed along the cross-sectional area of the helix increasing along the axis A from the distal end to the proximal end. The tapered helix may or may not have a gap between segments or loops of the helix when seen in a side view (e.g., the views of FIGS. 6A and 6B). FIG. 6C shows an end view of the ribbon 306 in the unstressed state, with an external surface of the latch 306f being visible (e.g., looking from the distal end toward the proximal end of the ribbon 306. FIG. 6D shows an end view of the ribbon 306 in the unstressed state, with an internal surface of the latch 306f being visible (e.g., looking into the tapered helix from the proximal end toward the distal end of the ribbon 306). An advantageous aspect of the ribbon 306 having a tapered structure is that such a structure may facilitate manufacturing of the cartridge assembly 100. With a tapered structure, the ribbon 306 may be easily collapsed into a coil by applying an axial force to squeeze the distal end and the proximal end toward each other (e.g., by a pinching action between two fingers), and may facilitate insertion of the coil onto the spindle 302a and into the ribbon chamber 110. For example, the tapered helix may collapse to form a coil when a first end of the tapered helix is placed against a flat surface (e.g., a table) and an axial force is exerted on a second end towards the flat surface. Materials of the ribbon 306 and molding techniques may be applied to result in a desired tapering profile.

According to some embodiments of the present technology, the rib latches 306c of the ribbon 306, discussed above, may protrude from one of the two surfaces of the ribbon 306. In some embodiments, the rib latches 306c may protrude from a surface forming an interior surface of the tapered helix, as shown in FIGS. 6A, 6B, and 6C. The rib latches 306c may be configured to engage with and slide along the spindle ribs 302b when the ribbon 306 is axially expanding or extending. In some embodiments, as discussed above, the rib latches 306c of the innermost loop of the coil (e.g., the innermost loop of the retracted portion 306a of the ribbon 306) may be in contact with the spindle ribs 302b and may be rotated by rotation of the spindle ribs 302b to form a growing or elongating helix. The innermost loop of the coil may be a continuously changing portion of the ribbon 306 as the helix elongates. The rib latches 306c that slide along the spindle ribs 302b may slide off the spindle ribs 302 consecutively as the helix elongates. Similarly, the rib latches 306c of the retracted portion 306a of the ribbon 306 may engage with the spindle ribs 302b consecutively as the helix elongates. In some embodiments, computerized control of an amount of rotation of the drive mechanism 302 may cause the ribbon 306 to be expanded to one or more predetermined positions, with each predetermined position corresponding to a known amount (or an known incremental amount) of the fluid being ejected out of the fluid chamber 158 relative to a previous predetermined position.

According to some embodiments of the present technology, a groove 310 may be provided on a surface of the ribbon 306 opposite to the surface on which the rib latches 306c are provided. In some embodiments, the groove 310 may be provided on an exterior surface of the ribbon 306, as shown in FIGS. 6A and 6B. The groove 310 may be configured to slide along a protruding bump portion 110a proximate a proximal end of the helix guide 112. In some embodiments, movement of the ribbon 306 along the bump portion 110a and along the spindle ribs 302b may cause the elongating helix to be columnar or cylindrical, which structurally may provide the helix with enhanced rigidity against the piston 156 while pushing against the piston 156. In some other embodiments, movement of the ribbon 306 along the bump portion 110a and along the spindle ribs 302b may cause the elongating helix to be tapered or conical. In some embodiments, the bump portion 110a may be a helical ridge 111a, 111a′ on an internal surface of the ribbon container 116, 116′. The helical ridge 111a, 111a′ may fit in and slidably engage with the groove 310 during elongation of the helix.

The edges of the ribbon may include complementary coupling features allowing adjacent edges of the ribbon to mate when the helical configuration is formed and the ribbon is axially extending, and, in some embodiments, to decouple from one another when transitioning to the coil configuration when the ribbon is being retracted. According to some embodiments of the present technology, the ribbon 306 may comprise a plurality of pegs 306d positioned along a first edge of the ribbon 306, and may comprise a plurality of holes 306e positioned along a second edge opposite to the first edge of the ribbon. In some embodiments, the pegs 306d may protrude from the surface forming the interior surface of the ribbon 306, as depicted in FIGS. 6A, 6B, and 6C. That is, the pegs 306d and the rib latches 306c may protrude from the same surface of the ribbon 306, with the rib latches 306c extending longitudinally from an edge region near the pegs 306c to the second edge of the ribbon 306. The pegs 306d and the holes 306e may be configured such that, when the ribbon 306 is expanding or extending, an increasing number of the pegs are received in an increasing number of the holes to form the helix such that each loop of the helix is interlocked with at least one adjacent loop of the helix.

While FIGS. 6A and 6B shows the ribbon 306 in an extended configuration forming a tapered helix, it should be appreciated that this is for illustrative purposes to show aspects of the ribbon 306, which may allow the ribbon 306 to unwind from the coil in a zipper-like configuration to form the helix, so that the ribbon 306 unwinds in a structured manner on itself, with lower edges of loops of the helix (e.g., formed of a lower edge of the ribbon 306) attaching to upper edges of the loops of the helix (e.g., formed an upper edge of the ribbon 306) as the ribbon 306 expands, as discussed herein. In some embodiments, the ribbon 306 may be manufactured and deployed in the cartridge assembly 100 as shown in FIG. 1, with the ribbon 306 being wound around itself such that some, most, or all of the ribbon 306 is retracted in a coil having a height corresponding to an edge-to-edge distance of the ribbon 306 (e.g., without requiring a force to be exerted on the ribbon 306 to maintain the ribbon 306 in the coil). As a result, in an initial configuration (e.g., prior to use of the dispenser 10 to administer any of the fluid in the fluid chamber 158), some, most, or all of the ribbon 306 may be retracted and wound around itself in the coil. As the ribbon 306 extends and joins upon itself to form a helix, as discussed herein, a structured and tapered shape may result or a columnar shape may result, each of which may provide accurate and effective dosing through accurate expansion of the helix, as also discussed herein.

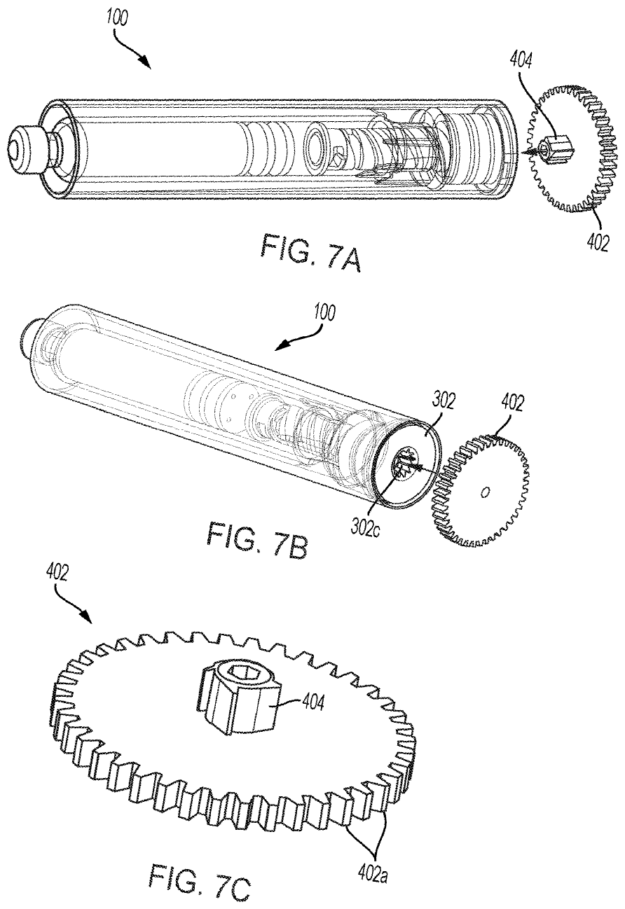

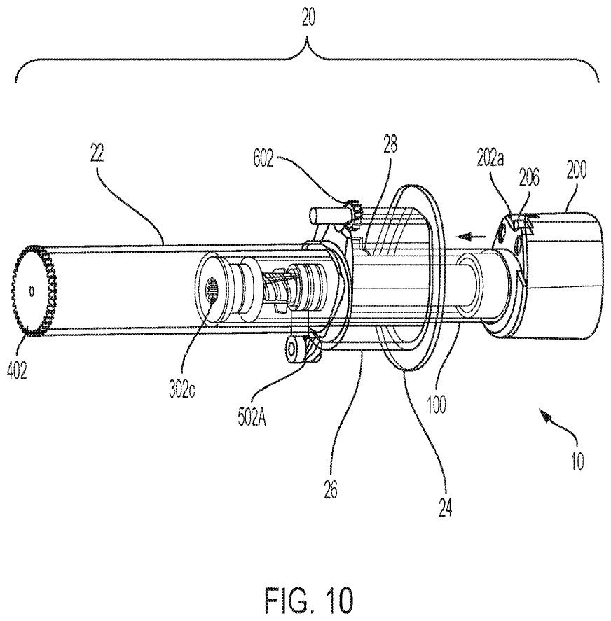

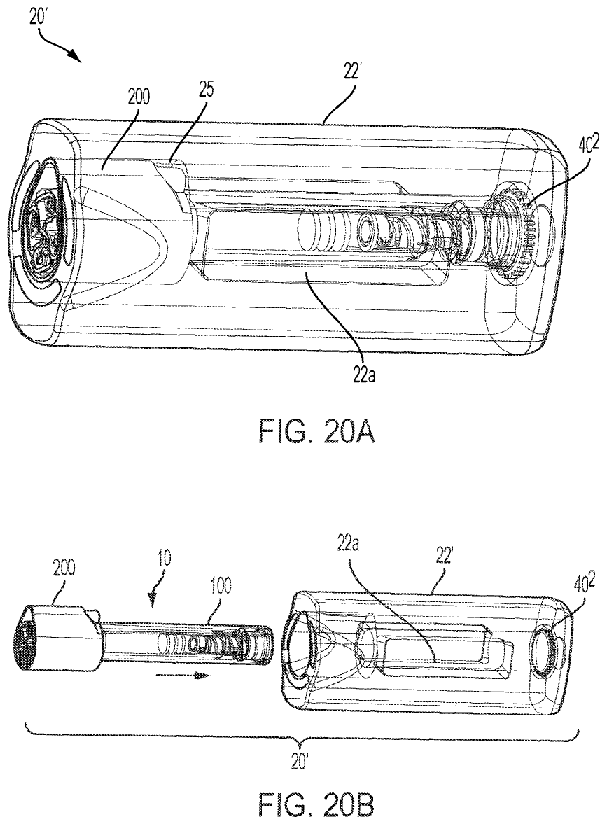

FIGS. 7A and 7B show perspective views of the cartridge assembly 100 and a driver gear 402 configured to rotationally drive the driven mechanism 302 of the drive assembly 300, according to some embodiments of the present technology. FIG. 7C shows a perspective view of the driver gear 402. In some embodiments, the driver gear 402 may be included as part of a cassette and may be incorporated in a housing of the cassette, as depicted in FIGS. 10, 20A, and 20B. In some embodiments, the driver gear 402 may comprise a disk-shaped body having a driver-gear diameter and gear teeth circumferentially spaced relative to one another along the circumferential edge surface of the disk shaped body. In some embodiments, the driver gear 402 may comprise a spline shaft 404 extending from an axially facing surface of the driver gear 402. The spline shaft 404 may be configured to fit into the recess 302c in the surface of the driven mechanism 302, and the spline shaft 404 may have teeth configured to engage with the recessed teeth 302d of the recess 302c. The recess 302c and a body of the spindle 302a may have a coaxial relationship along the longitudinal axis A, as shown in FIGS. 7A and 7B. In some embodiments, the driver gear 402 may be a spur-type gear configured to be driven to rotate via a force applied to teeth 402a of the driver gear 402. Rotation of the driver gear 402 may drive rotation of the driven mechanism 302 via the spline shaft 404 of the driver gear 402 and the recess 302c and teeth 302d of the driven mechanism 302. In some embodiments, when the cartridge assembly 100 is inserted into a housing of a cassette (e.g., axially inserted as shown in FIG. 10), the spline shaft 404 may be located along the longitudinal axis A to facilitate an insertion coupling of the spline shaft 404 with the recess 302c of the cartridge assembly 100, which may be located along the longitudinal axis A. The driver gear 402 and the driven mechanism 302 may be arranged together and may advantageously reduce an amount of thrust experienced by the ribbon 306 and by the cassette. In some embodiments, the driver-gear diameter of the body of the driver gear 402 may be at least greater than a spindle-base diameter of the spindle base 302e of the spindle 302a.

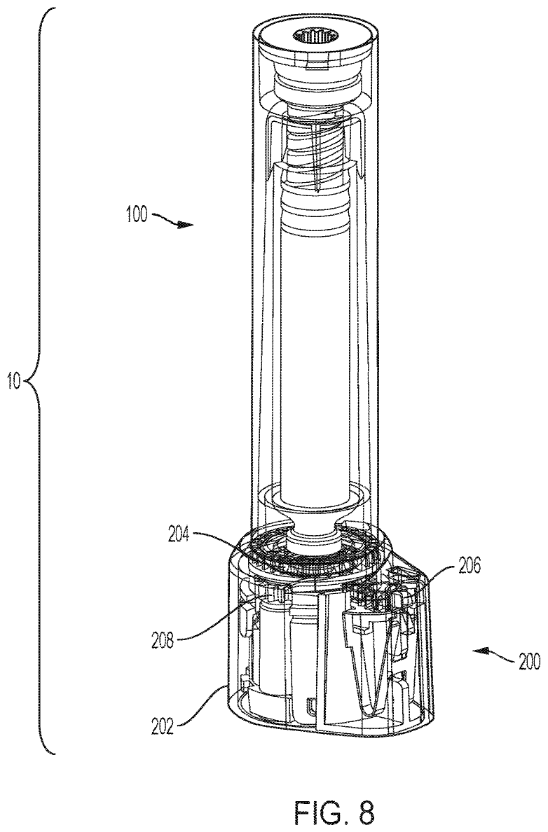

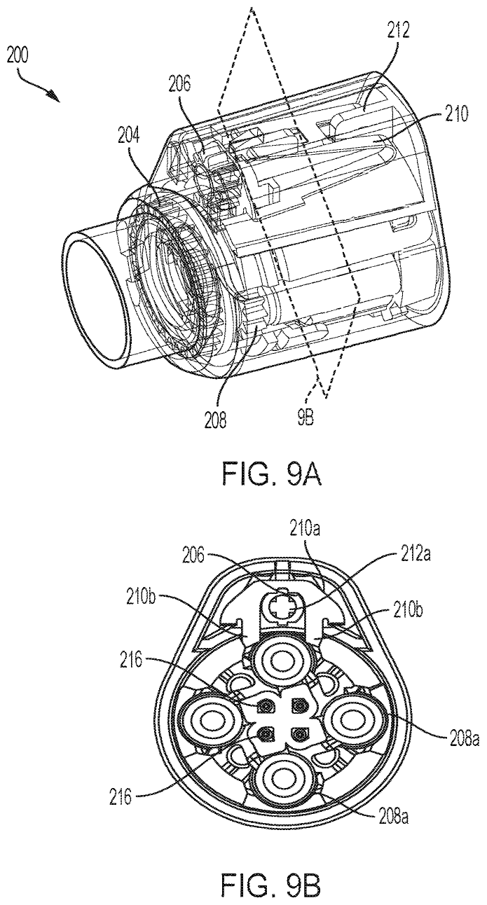

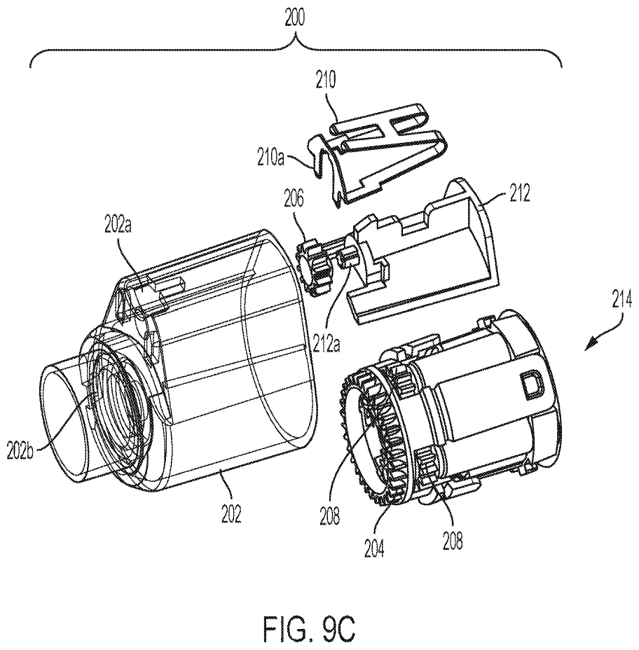

FIG. 8 shows a perspective view of the dispenser 10, according to some embodiments of the present technology. In FIG. 8, a housing 202 of the needle assembly 200 is depicted as translucent so that internal components may be seen. FIG. 9A shows a perspective view of the needle assembly 200, and FIG. 9C shows a perspective view of the needle assembly 200 in a partially disassembled state. FIG. 9B shows a plan view of a cross-section of the needle assembly 200 cut along a plane 9B in FIG. 9A.

According to some embodiments of the present technology, the needle assembly 200 may be attached to the distal end of the cartridge assembly 100, such that the septum 152 of the cartridge assembly 100 is received in a recess of the needle assembly 200. In some embodiments, during an injection process, a piercer of the needle assembly 200 may be driven to pierce the septum 152 to enable the fluid in the fluid chamber 158 be ejected out of the fluid chamber 158. For example, the septum 152 may be pierced according to techniques described in International Application No. PCT/US2021//063178 entitled “Fluid Delivery System with Needle Assembly” or in International Application No. PCT US2021/063180 entitled “Fluid Delivery System with Needle Assembly,” each of which is incorporated by reference herein in its entirety. Examples of a housing of the medication delivery device that may be coupled to the dispenser 10 may be found in these International Applications. In some embodiments in which the fluid is a liquid medication, the fluid ejected from the fluid chamber 158 may be injected into a patient through a needle 216 of the needle assembly 200. The needle assembly 200 may carry a single needle or a plurality of needles (e.g., two needles, three needles, four needles, etc.), as discussed herein.

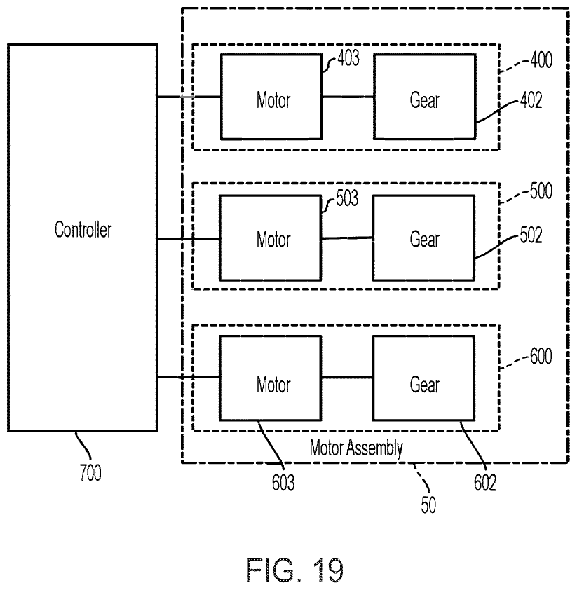

According to some embodiments of the present technology, the needle assembly 200 may comprise a plurality of needles 216 that may be individually activated for use. The needle assembly 200 may comprise a needle-selection gear 204, which may be driven to move a selected one of the needles 216 to an activated position. The selected needle 216 may be a next one of the plurality of needles 216 or may be a particular one of the plurality of needles 216. The selected needle 216 may be rotated into the activated position by rotation of the needle-selection gear 204 through action of a driver assembly 600 (FIG. 19), discussed below. In some embodiments, each needle 216 of the needle assembly 200 may be operatively connected to a needle-movement gear 208 and a gear assembly 208a, which may be configured to move the needle 216 axially from a retracted position in the housing 202, at which a needle tip is retracted from a base surface 202c at a distal end of the housing 202, to an injection position, at which the needle tip extends beyond the base surface 202c (e.g., into a patient). If a needle 216 is not in the activated position, the needle 216 cannot move from the retracted position to the injection position.

According to some embodiments of the present technology, when a needle 216 is in the activated position (“activated needle”), a transfer gear 206 of the needle assembly 200 may drive movement of the needle 216 to and from the injection position and the retracted position. The transfer gear 206 also may be referred to herein as an idler 206. In some embodiments, when in the activated position, teeth of the needle-movement gear 208 of the activated needle 216 may be intermeshed with teeth of the transfer gear 206, such that rotation of the transfer gear 206 may drive rotation of the needle-movement gear 208. The transfer gear 206 may be driven to rotate through action of a driver assembly 400 (FIG. 19), discussed below. Rotation of the needle-movement gear 208 by the transfer gear 206 may cause the activated needle 216 to move axially from the retracted position to the injection position.

The inventors have recognized and appreciated that safety is an important consideration in use of the dispenser 10, especially when the dispenser 10 is to be used by lay persons who are not trained in handling needles and/or injection fluids (e.g., liquid medications). The inventors have therefore provided gear-lock mechanisms for the dispenser 10. The gear-lock mechanisms may prevent the needle-selection gear 204 from placing any of the needles 216 in the activated position and/or may prevent the transfer gear 206 from moving any of the needle-movement gears 208 unless certain unlocking features are satisfied, as discussed herein.

One such gear-lock mechanism is a lock spring 210, which may comprise a U-shaped spring face 210a configured to prevent rotation of the needle-selection gear 204 and to prevent rotation of the transfer gear 206 when the lock spring 210 is in a locked position. FIG. 9B shows a surface of the needle assembly 200 on the left of the plane 9B (see FIG. 9A), with the lock spring 210 in the locked position, according to some embodiments of the present technology. When the lock spring 210 is in the locked position, projections 210b of the spring face 210a are lodged in spaces in the needle assembly 200, preventing rotation of the needle-selection gear 204. For example, the projections 210b may straddle one of the gear assemblies 208a, as shown in FIG. 9B, or may be inserted between two of the gear assemblies 208a, such that the projections 210b block rotation of the needle-selection gear 204 and may block movement of a needle 216 into the activated position. Additionally, when the lock spring 210 is in the locked position, a base of the spring face 201a may abut against the transfer gear 206 to prevent rotation of the transfer gear 206. For example, a protrusion on a stem portion of the transfer gear 206 may be seated in a recess in the base of the lock spring 210 such may that the transfer gear 206 may not rotate. In some embodiments, the lock spring 210 and the transfer gear 206 may be mounted on a support frame 212 of the needle assembly 200. The support frame 212 may be attached to a needle subassembly 214 comprised of the needles 216, the needle-selection gear 204, and the needle-movement gears 208, and may be positioned such that the transfer gear 206 may rotate the needle-movement gear 208 of a needle 216 in the activated position when the lock spring 210 is in an unlocked position. In some embodiments, the transfer gear 206 may be movably mounted on a frame post 212a of the support frame 212, and the projections 210b of the spring face 210a may straddle the transfer gear 206 and the frame post 212a, as depicted in FIG. 9B. As discussed below, when the lock spring 210 is moved from the locked position to the unlocked position, the base of the spring face 210a may be shifted away from the transfer gear 206, and the projections 210b of the spring face may be shifted away from the gear assemblies 208a, thus enabling the needle-selection gear 204 to rotate a selected one of the needles 216 into the activated position adjacent the transfer gear 206 and also enabling the transfer gear 206 to rotate and drive the needle-movement gear 208 of the activated needle 216.

FIG. 10 shows a perspective view of a cassette 20 comprising the dispenser 10 partially inserted in a cassette housing 22, according to some embodiments of the present technology. In some embodiments, when the needle assembly 200 and the cartridge assembly 100 are assembled together to form the dispenser 10 but the dispenser 10 is not installed in the cassette housing 22, the lock spring 210 is in the locked position. This may serve as a safety mechanism that prevents accidental movement of the needles 216 into the injection position. In some embodiments, the cassette housing 22 may comprise a compartment 26 configured to receive the needle assembly 200. When the dispenser 10 is fully inserted in a cavity of the cassette housing 22, a portion of the compartment 26 may cause the lock spring 210 to move into the unlocked position.

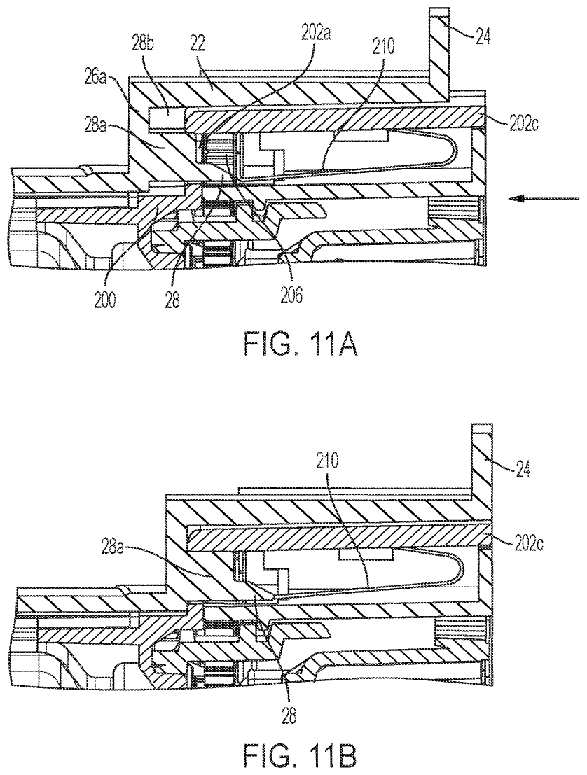

FIG. 11A shows a perspective view of a partial longitudinal cross-section of the needle assembly 200 partially inserted in the compartment 26 of the cassette housing 22, according to some embodiments of the present technology. Because the needle assembly 200 is not fully inserted in the compartment 26, the lock spring 210 of the needle assembly 200 may be in the locked position. In some embodiments, when a base 24 of the cassette housing 22 is not aligned with a base 202c of the housing 202 of the needle assembly 200, the needle assembly 200 may not be fully inserted in the compartment 26 such that an internal recess 28b of the compartment 26 is not engaged with an edge portion of the housing 202, as depicted in FIG. 11A. An arrow in FIG. 11A shows a direction of movement of the needle assembly 200 relative to the cassette housing 22 during installation of the dispenser 10 into the cassette housing 11. The compartment 26 may comprise a base 26a supporting a tang base 28a and a tang 28 protruding axially from the tang base 28a. The tang 28 may be configured to engage with the lock spring 210 to move the lock spring 210 from the locked position to the unlocked position when then needle assembly 200 is fully inserted in the compartment 26 of the cassette housing 22. FIG. 11B shows the needle assembly 200 fully inserted in the compartment 26 of the cassette housing 22, such that the base 24 of the cassette housing 22 is coplanar with the base 202c of the housing 202 of the needle assembly 200. When fully inserted, the tang 28 extending from the base 26a of the compartment 26 may push against a portion of the lock spring 210 to compress the lock spring 210 and move the lock spring 210 such that the base of the spring face 210a may be shifted away from the transfer gear 206 and the projections 210b of the spring face 210a may be shifted away from the gear assemblies 208a, thus placing the lock spring 210 in the unlocked state. Such shifting enables the needle-selector gear 204 to rotate, thus enabling a selected one of the needles 216 to be moved to the activated position and also enabling the transfer gear 206 to rotate to drive the needle-movement gear 208 of the activated needle 216.

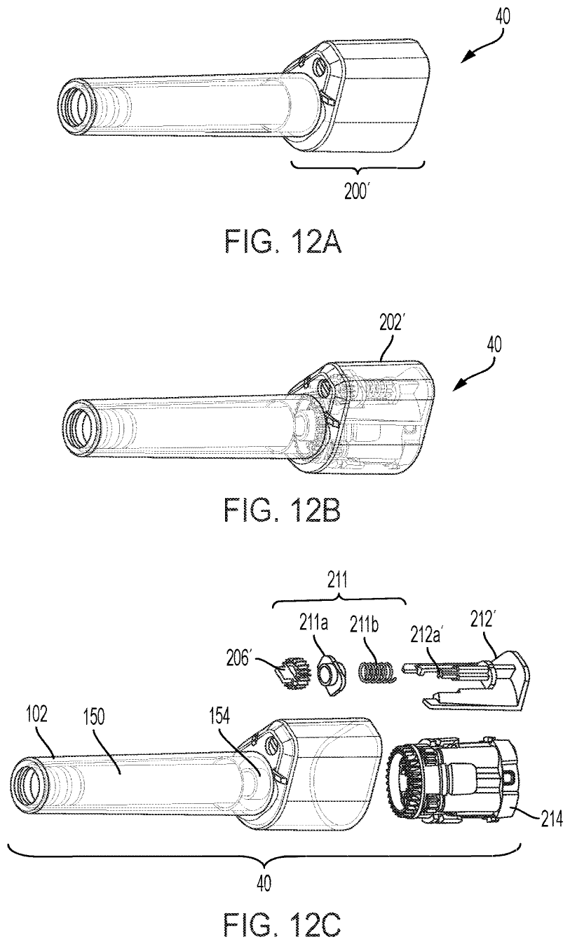

FIG. 12A shows a perspective view of a distal section of a dispenser 40, according to some embodiments of the present technology. The dispenser 40 may be similar in many respects to the dispenser 10 described above. Portions of the dispenser 40 that are the same as the dispenser 10 may be represented by the same reference numerals. Portions of the dispenser 40 that are modifications of the dispenser 10 may be represented by modified versions of the same references numerals (e.g., with the addition of ′ or ″ after the reference numeral). In some embodiments, the gear-lock mechanism of the dispenser 40 may be different from the gear-lock mechanism of the dispenser 10. FIG. 12B shows the same view as FIG. 12A but with the housing 202′ of the needle assembly 200′ of the dispenser 40 appearing translucent. FIG. 12C shows a perspective view of the distal section of the dispenser 40 in a partially disassembled state, according to some embodiments of the present technology.

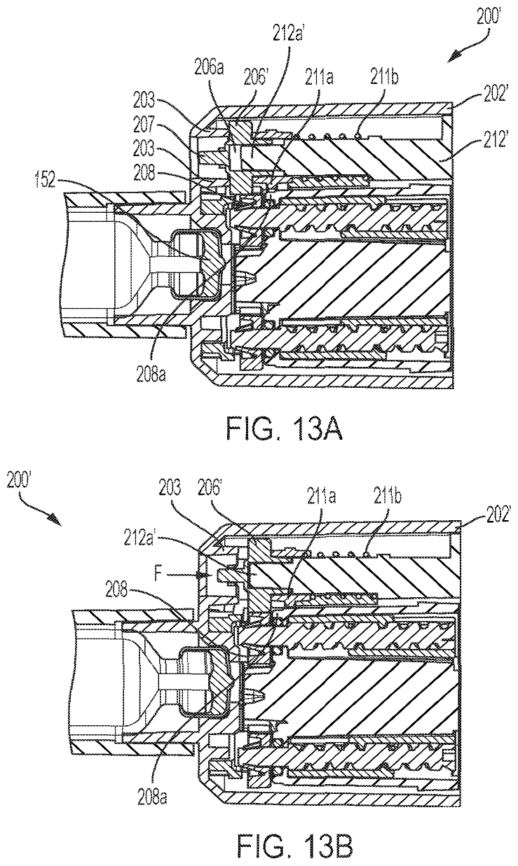

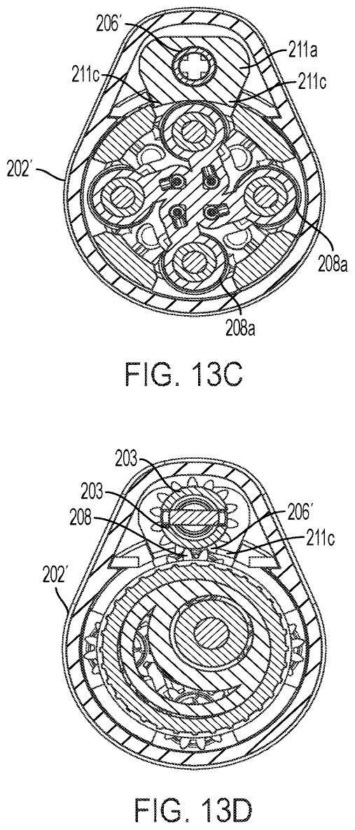

According to some embodiments of the present technology, the gear-lock mechanism of the dispenser 40 may comprise a lock spring 211, which may comprise a lock plate 211a and a coil spring 211b configured to prevent rotation of the needle-selection gear 204 and to prevent rotation of the transfer gear 206′ when the lock spring 211 is in a locked position. FIG. 13A shows a view of a longitudinal cross-section of the needle assembly 200′ in a locked state, and FIG. 13B shows the same view of the needle assembly 200′ in an unlocked state, according to some embodiments of the present technology. In some embodiments, the lock spring 211 and the transfer gear 206′ may be movably mounted on the needle subassembly 214 via the support frame 212′. The coil spring 211b, the lock plate 211a, and the transfer gear 206′ may be movably mounted on the frame post 212a′ of the support frame 212′ such that the coil spring 211b biases the lock plate 211 and the transfer gear 206′ in the unlocked position. In the unlocked position, an end of the frame post 212a′ may be partially disposed in a cavity 206a of the transfer gear 206′. As depicted in FIG. 13A, the coil spring 211b when at rest may bias the lock plate 211a against the transfer gear 206a′ such that the transfer gear 206′ is pushed against ribs or lips 203 protruding from the housing 202′, preventing the transfer gear 206′ from rotating. In this position, the transfer gear 206′ may not be fully meshed with the needle-movement gear 208 of the activated needle 216, as depicted in FIG. 13A. Additionally, when the coil spring 211b is at rest, the lock plate 211a may bear against portions of the needle subassembly 214, preventing rotation of the needle-selection gear 204. FIG. 13C shows a plan view of a lateral cross-section of the needle assembly 200′ cut through the lock plate 211a when the lock spring 211 is in the locked state, showing blocking potions 211c of the lock spring 211 extending into a portion of the needle subassembly 214 and bearing against surfaces of the needle subassembly 214, thus preventing rotation of the needle-selection gear 204. FIG. 13D shows a plan view of a lateral cross-section of the needle assembly 200′ cut through the transfer gear 206′ when the lock spring 211 is in the locked stated, showing the lip 203 of the housing 202′ blocking rotational movement of the transfer gear 206′.

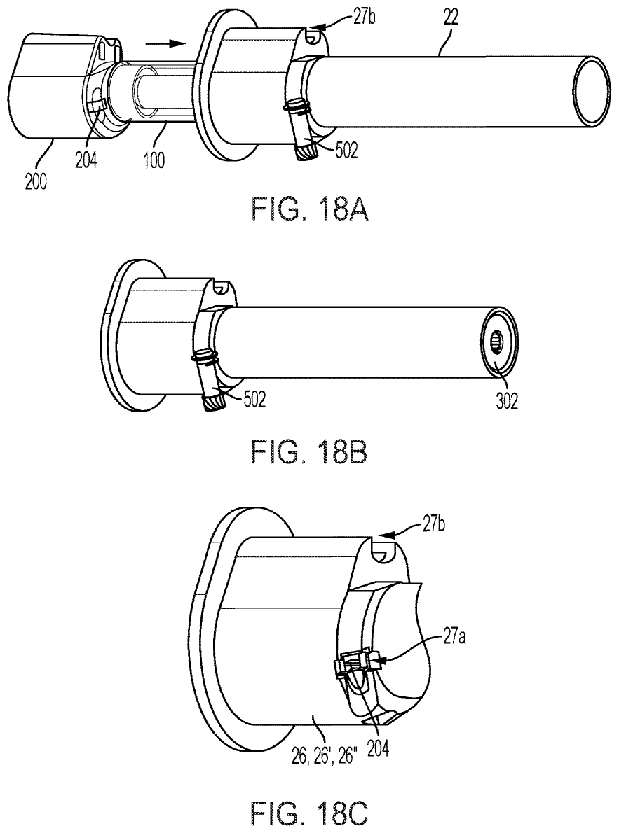

According to some embodiments of the present technology, when an axial force is applied to the lock spring 211, the lock spring may be moved from the locked position to the unlocked position. A direction of the axial force is represented by an arrow F in FIG. 13B. The axial force may compress the coil spring 211b and may enable axial movement of the lock plate 211a away from blocking rotation of the needle-selection gear 204 and may enable axial movement of the transfer gear 206′ away from the lip 203, thus enabling the needle-selection gear 204 to rotate and also enabling the transfer gear 206′ to rotate. In some embodiments, the axial force may be applied by a driver gear 602, which may be mountable on the cassette housing 22 before or after the dispenser 40 is fully installed in the cassette housing 22. The driver gear 602 may extend through an opening 27b in the cassette housing (see FIGS. 10, 18A and 18C) and may exert the axial force on the transfer gear 206′ to push the transfer gear 206′ away from the lip 203, in the direction of the arrow F. The transfer gear 206′ may, in turn, push the lock plate 211a in the direction of the arrow F, causing the lock plate 211a to move away from blocking movement of the needle-selection gear 204. In some embodiments, the driver gear 602 may engage with a gear handle 207 (e.g., a slot, a protruding bar, etc.) of the transfer gear 206′ to push the transfer gear 206′ and/or to rotate the transfer gear 206′. The driver gear 602 may include a portion with a shaft configuration, although other gear configurations are contemplated. In some embodiments, the driver gear 602 may be part of a motor assembly, as discussed below. The cassette 20, 20′ and the motor assembly may be part of a medication delivery device and may be housed at least partially in a housing of the medication delivery device.

As will be appreciated, application of a force on the transfer gear 206′ in the direction of the arrow F may push the needle assembly 200′ (and the dispenser 40) out of the cassette housing 22. In a related manner, a natural state of the lock spring 210 may prevent the dispenser 10 shown in FIGS. 10, 11A, and 11B from staying in the fully inserted position (FIG. 11B) and may cause the dispenser 10 to be in a partially inserted position in the cassette housing 22 (FIG. 11A) unless a retaining force is applied to retain the dispenser 10 at the fully inserted position in the cassette housing 22.

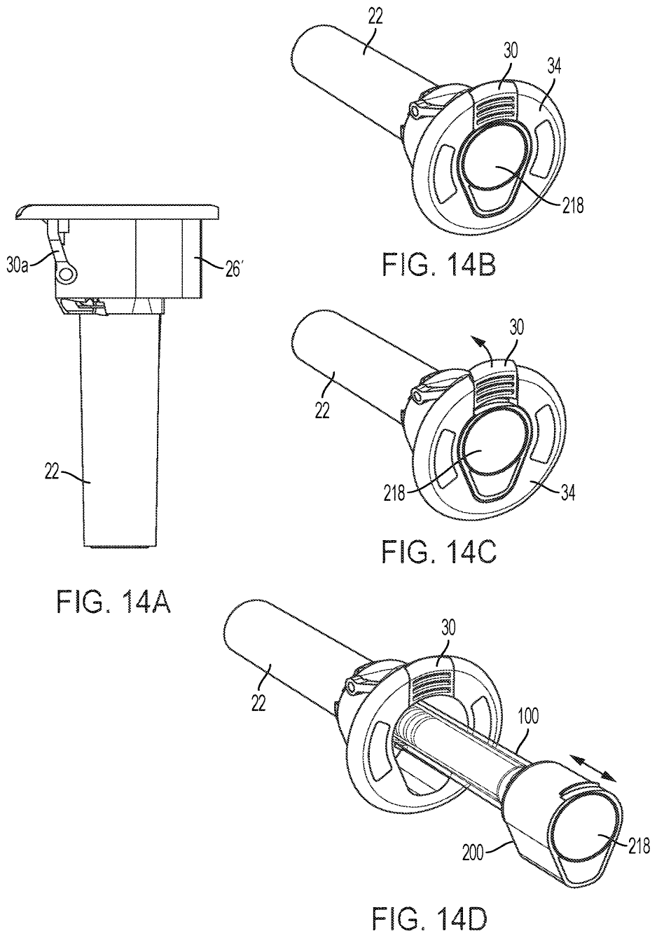

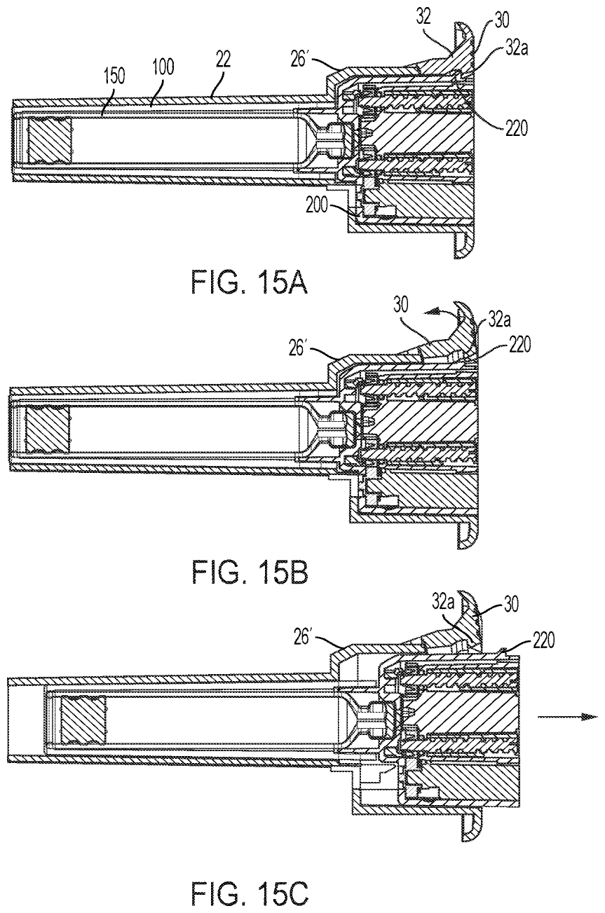

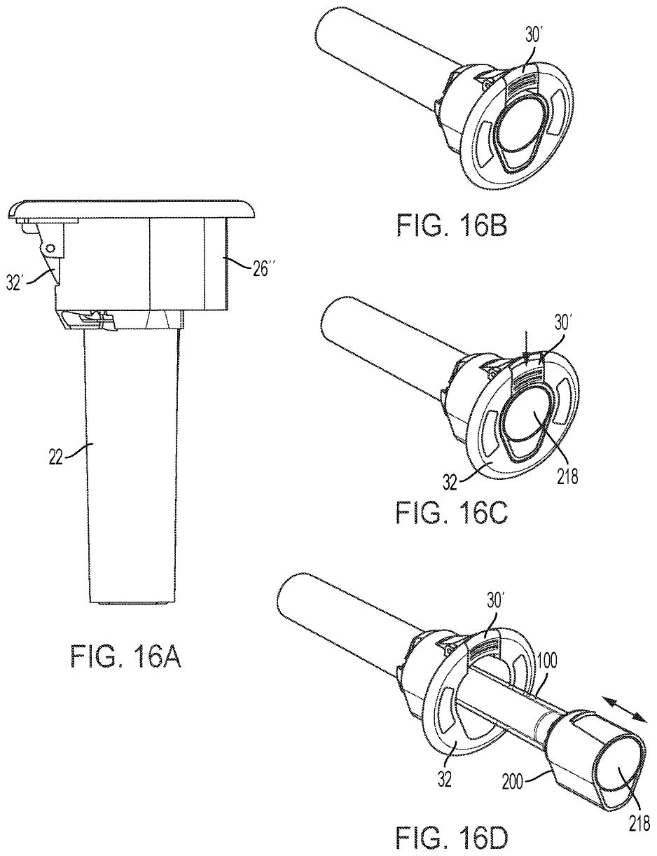

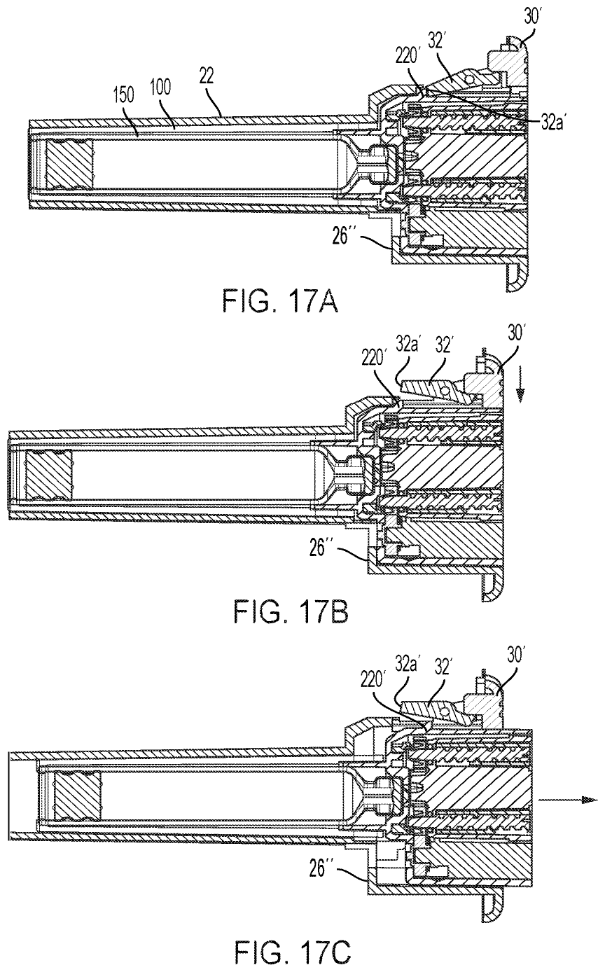

To keep the dispenser 10, 40 in place in the cassette housing 22, a latch mechanism may be used. FIGS. 14A through 14D and 15A through 15C show views of a first type of latch mechanism that may be used, and FIGS. 16A through 16D and 17A through 17C show views of a second type of latch mechanism that may be used, according to some embodiments of the present technology.

The first type of latch mechanism may comprise a latch 32 configured to engage with a latch receiver 220, according to some embodiments of the present technology. The latch 32 may be movably attached to the compartment 26′ of the cassette housing 22 via a connector 30a. The latch receiver 220 may comprise a protruding ledge on an external surface of the housing 202, 202′ of the needle assembly 200, 200′. The latch 32 may comprise an engagement surface 32a configured to abut the ledge of the latch receiver 220 to retain the needle assembly 200, 200′ in the compartment 26′ when in a latched position, as schematically depicted in FIG. 15A. The latch 32 also may comprise an actuator 30, which may be manipulated by a user to release the latch 32 from engagement with the latch receiver 220. In some embodiments, the actuator 30 and the latch 32 may be formed as a single structure, with the engagement surface 32a of the latch 32 positioned on an internal surface of the structure and with the actuator 30 positioned on an external surface of the structure. In some embodiments, the latch 32 may be disengaged by the user by sliding the actuator 30 in a lateral release direction (or in a direction having a lateral component) parallel to a contact surface 34 of the cassette housing 22. For example, FIGS. 14C and 15B show arrows indicating directions of movement of the actuator 30 to release or disengage the latch 32. In some embodiments, a surface of the actuator 30, the contact surface 34 of the cassette housing 22, and a contact surface 218 of the needle assembly 200, 200′ may be generally coplanar, such that when the contact surface 218 of the needle assembly 200, 200′ is placed against an injection surface (e.g., a skin surface of a patient to be injected with the fluid in the fluid chamber 158) the actuator 30 may not be manipulated in the lateral release direction. Such a coplanar structure may advantageously prevent accidental release or disengagement of the latch 32 by the user during an injection process. In some embodiments, the latch 32 and the latch receiver 220 may comprise complementary angled surfaces configured to slide against each other during insertion of installation of the needle assembly 200, 200′ into the compartment 26′ of the cassette housing 22. When the needle assembly 200, 200′ is fully inserted in the compartment 26′, the latch 32 may snap into the latched position when the latch receiver 220 falls into a recess of the latch 32 and the engagement surface 32a of the latch 32 abuts the latch receiver 220, as depicted in FIG. 15A. In some embodiments, the connector 30a may be a spring that biases the latch 32 such that, once the needle assembly 200, 200′ and the compartment 26′ of the cassette housing 22 are in the latched position, the latch 32 remains latched to the latch receiver 220 until the actuator 30 is manipulated in the lateral release direction, as discussed above. In some embodiments, upon manipulation of the actuator 30 to release or disengage from the latch receiver 220 of the needle assembly 200, 200′, a spring force of the lock spring 210, 211 of the needle assembly 200, 200′ may bias the dispenser 10, 40 to eject the dispenser 10, 40 from the cassette housing 22, as depicted in FIG. 15C.

The second type of latch mechanism may comprise a latch 32′ configured to engage with the latch receiver 220′, according to some embodiments of the present technology. The latch 32′ may be pivotably attached to the compartment 26″ of the cassette housing 22 via a hinge-type connector (not shown). The latch 32′ may comprise an engagement surface 32a′ configured to abut the ledge of the latch receiver 220′ to retain the needle assembly 200, 200′ in the compartment 26″ when in the latched position, as schematically depicted in FIG. 17A. The latch 32′ also may comprise an actuator 30′, which may be manipulated by a user to release the latch 32′ from engagement with the latch receiver 220′. In some embodiments, the actuator 30′ may be configured to pivot the latch 32′ from the latched position (FIG. 17A) to a release position (FIG. 17B). In some embodiments, the latch 32′ may be placed in the release position by the user by sliding the actuator 30′ in a lateral direction (or in a direction having a lateral component) parallel to the contact surface 34 of the cassette housing 22. For example, FIGS. 16C and 17B show arrows indicating a direction of movement of the actuator 30′ to release or disengage the latch 32′. In some embodiments, the surface of the actuator 30′, the contact surface 34 of the cassette housing 22, and a contact surface 218 of the needle assembly 200, 200′ may be generally coplanar, such that when the contact surface 218 of the needle assembly 200, 200′ is placed against an injection surface the actuator 30′ may not be manipulated to cause the latch 32′ to pivot to the release position, to prevent accidental release or disengagement of the latch 32′ during an injection process. In some embodiments, the actuator 30′ may comprise a spring that biases the actuator 30′ such that when the needle assembly 200, 200′ and the compartment 26″ of the cassette housing 22 are in the latched position the latch 32′ remains latched to the latch receiver 220′ until the actuator 30′ is manipulated to pivot the engagement surface 32a′ of the latch 32′ away from the latch receiver 220′, as discussed above. In some embodiments, upon manipulation of the actuator 30′ to release or disengage from the latch receiver 220′ of the needle assembly 200, 200′, a spring force of the lock spring 210, 211 may bias the dispenser 10, 40 to eject the dispenser 10, 40 from the cassette housing 22, as depicted in FIG. 17C.

FIG. 18A shows a view of the cassette housing 22 during insertion of the dispenser 10 into the cassette housing 22, and FIG. 18B shows a view after the dispenser 10, 40 is fully inserted in the cassette housing 22, according to some embodiments of the present technology. In FIGS. 18A and 18B, a driver gear 502 for rotating the needle-selection gear 204 of the needle assembly 200 of the dispenser 10 is positioned at an opening 27a in the compartment 26, 26′, 26″ through which the driver gear 502 may contact and rotate the needle-selection gear 204. In some embodiments, the driver gear 502 may include a portion with a worm-gear configuration, although other gear configurations are contemplated. In some embodiments, the driver gear 502 may be part of a driver gear assembly 502A (FIG. 10) comprising at least one other gear, and the driver gear assembly 502A may be incorporated in a housing of a medication delivery device, as discussed herein. FIG. 18C shows an enlarged of the compartment 26, 26′, 26″ of the cassette housing 22, depicting teeth of the needle-selection gear 204 through the opening 27a. Although not fully visible in FIGS. 18A through 18C, an opening 27b is provided in the compartment 26, 26′, 26″ to enable the driver gear 602, discussed above, to contact and rotate the transfer gear 206, 206′ of the dispenser 10, 40. As discussed above, in some embodiments, when the dispenser 40 is fully inserted in the cassette housing 22 and the driver gear 602 is mounted to the cassette housing 22, the driver gear 602 may exert a force on the transfer gear 206′ that may move the lock spring 211 to the unlocked position.

FIG. 19 shows a block diagram of a motor assembly 50 comprising a plurality of driver assemblies 400, 500, 600. The motor assembly 50 may be coupled to a controller 700 configured to drive each of the driver assemblies 400, 500, 600. In some embodiments of the present technology, the controller 700 may comprise at least one computer processor (e.g., a CPU) programed to control the driver assemblies 400, 500, 600 individually or in a coordinated manner with each other. In some embodiments, the driver assembly 400 may comprise the driver gear 402, discussed above, for driving rotation of the driven mechanism 302 of the drive assembly 300. The driver assembly 400 may comprise a motor 403 configured to cause rotation of the driver gear 402. Similarly, in some embodiments, the driver assembly 500 may comprise the driver gear 502, discussed above, for driving rotation of the needle-selection gear 204. The driver assembly 500 may comprise a motor 503 configured to cause rotation of the driver gear 502. In some embodiments, the driver assembly 600 may comprise the driver gear 602, discussed above, for driving rotation of the transfer gear 206, 206′. The driver assembly 600 may comprise a motor 603 configured to cause rotation of the driver gear 602. In some embodiments, the controller 700 may be provided with an actuator (not shown) that enables a user to initiate an injection process. For example, after the user has placed the cassette in an injection position (e.g., against the user's own skin surface or against a skin surface of a patient), the user may manipulate the actuator to cause an automated and coordinated movement of any one of or any combination of: (1) a needle 216 of the needle assembly 200, 200′ to be positioned in the activated position through rotation of the needle-selection gear 204 via the driver assembly 600; (2) movement of the drive assembly 300 to cause movement of the piston 156 towards the septum 152, to eject the fluid in the fluid chamber 158 by a predetermined amount corresponding to, e.g., an amount of rotation of the driven mechanism 302; and (3) movement of the transfer gear 206, 206′ to cause the activated needle 216 to move from a retracted position to an injection position and from the injection position to the retracted position. As noted above, movement of the transfer gear 206, 206′ also may cause the septum 152 to be pierced to permit the fluid to be ejected into the activated needle 216.

As will be appreciated, the cassette housing 22 may have a form different than the form shown in FIG. 10. FIG. 20A shows a view of the cassette 20′ in which the cassette housing 22′ may be shaped generally rectangularly and sized to be easily grasped by a user. In some embodiments of the present technology, the cassette housing 22′ may have rounded edges and/or slightly curved surfaces, as shown. In some embodiments, the cassette housing 22′ may have an internal cavity shaped to receive the dispenser 10. For example, the internal cavity the cassette housing 22′ may have a generally cylindrical portion to receive the cartridge assembly 100 and a compartment 25 shaped to receive the needle assembly 200. FIG. 20B shows a view in which the dispenser 10 is being inserted in the cassette housing 22′ to form the cassette 20′. In some embodiments, the cassette housing 22′ may be configured with a recess to accommodate the driver gear 402. Optionally, a window 22a may be provided in the cassette housing 22′ to enable a position of the piston 156 and/or a level of the fluid to be visible. Although not shown in FIGS. 20A and 20B, the cassette housing 22′ may include openings for the driver gears 502, 602 to contact the needle assembly 200.

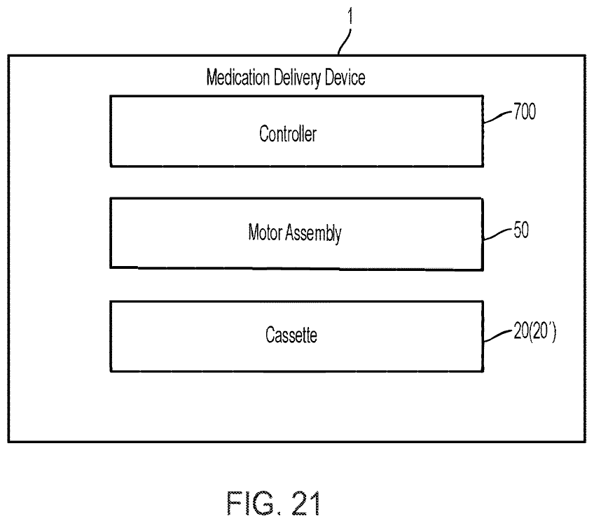

FIG. 21 shows a block diagram of a medication delivery device 1, according to some embodiments of the present technology. In some embodiments, the device 1 may comprise the cassette 20, 20′, the motor assembly 50, and the controller 700, some or all of which may be housed within or partially within a housing of the device 1. In some other embodiments, the controller 700 may be external to the housing of the device 1 and may be configured to control the motor assembly 50 remotely through control signals transmitted via a dedicated cable and/or through control signals transmitted wirelessly using known technology for wireless communications.

### Conclusion

It should be understood that various alterations, modifications, and improvements may be made to the structures, configurations, and methods discussed above, and are intended to be within the spirit and scope of the technology disclosed herein. Further, although advantages of the present technology are indicated, it should be appreciated that not every embodiment of the present technology will include every described advantage. Some embodiments may not implement any features described as advantageous herein. Accordingly, the foregoing description and attached drawings are by way of example only.

It should be understood that some aspects of the present technology may be embodied as one or more methods, and acts performed as part of a method of the present technology may be ordered in any suitable way. Accordingly, embodiments may be constructed in which acts are performed in an order different than shown and/or described, which may include performing some acts simultaneously, even though shown and/or described as sequential acts in various embodiments.

Various aspects of the present technology may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Various aspects are described in this disclosure, including the summary, which include, but are not limited to, the following aspects:

In various aspects of the disclosure, the force-transfer portion has a tapered profile and comprises at least one drive rib extending radially from a surface of the force-transfer portion.

In various aspects of the disclosure, the at least one drive rib, along an axial length of the drive rib, extends a radial distance (d) from a radially outer edge of the drive rib to the longitudinal axis.

In various aspects of the disclosure, a cartridge housing includes a first section configured to contain a least a portion of the axially extendible portion, a second section configured to contain at least a portion of the cartridge, and a third section configured to align the axially extendible portion and the cartridge.

In various aspects of the disclosure, the third section of the cartridge housing includes housing ribs configured to contact a proximal end of the cartridge and to position the cartridge relative to an internal wall of the cartridge housing.

In various aspects of the disclosure, the housing ribs extend radially from the internal wall of the cartridge housing and aligns the cartridge to a predetermined position in the cartridge housing, and the cartridge abuts shoulder portions of the housing ribs.

In various aspects of the disclosure, the third section of the cartridge housing includes a guide configured to center the axially extendible portion during movement from the retracted position to the at least one extended position.

In various aspects of the disclosure, a foot is attached to the distal end of the axially extendible portion, wherein the foot is configured to move in the second section of the cartridge housing, the foot includes a foot surface configured to come into contact with a piston surface of the piston to move the piston toward the distal opening of the cartridge, and the foot surface includes a relief structure configured to engage with at least one corresponding surface structure on the piston surface.

In various aspects of the disclosure, the force-transfer portion includes a rotatable spindle engaged with an internal loop of a coil formed of the axially extendible portion, and a spline portion configured to receive a spline-shaped force applicator.

In various aspects of the disclosure, the spline portion includes a spline-shaped recess configured to receive a spline shaft, and the spline-shaped recess and the rotatable spindle are in a coaxial relationship along the longitudinal axis.

In various aspects of the disclosure, the axially extendible portion includes a ribbon, a retracted portion the ribbon is configured to form the coil, the ribbon is configured to uncoil and extend controllably in response to the applied force, to form a helix having a controllable axial length, and the piston and the helix are coaxial.

In various aspects of the disclosure, the force-transfer portion includes a plurality of drive ribs extending radially from the surface of the force-transfer portion, the ribbon includes a plurality of rib latches protruding from a first surface of the ribbon, and the rib latches are configured to slidably engage along the drive ribs when the ribbon is extending from the retracted position to the at least one extended position.

In various aspects of the disclosure, an internal surface of the helix includes the first surface of the ribbon, a second surface of the ribbon includes a groove configured to movably engage with and slide along a bump portion of the cartridge housing, and in response to movement of the ribbon along the bump portion of the cartridge housing and along the drive ribs, the helix is formed from the ribbon to be columnar.

In various aspects of the disclosure, the ribbon includes mating elements along its edges, and in some examples, a plurality of pegs positioned along a first edge of the ribbon, and a plurality of holes positioned along a second edge of the ribbon, and, when the axially extendible portion is extending from the retracted position to the at least one extended position, an increasing number of the pegs are received in an increasing number of the holes such that the helix is interlocked to form a cylinder having a fixed diameter.

In various aspects of the disclosure, the pegs are positioned on the first surface of the ribbon, the holes are positioned on the second surface of the ribbon, and the rib latches extend longitudinally between the first and second edges of the ribbon.

In various aspects of the disclosure, the ribbon is formed of a resilient material such that, when not confined in the cartridge housing and in an absence of a force exerted on the ribbon, the ribbon forms a tapered helix having a diameter that varies from a first end of the tapered helix to a second end of the tapered helix, and the tapered helix collapses to form a coil when an axial force is exerted on the first end or the second end or the first and second ends of the tapered helix, the axial force being a force along an axis of the tapered helix.

In various aspects of the disclosure, the needle assembly is attached to a distal end of the cartridge housing, and the septum is positioned within a recess of the needle assembly.

In various aspects of the disclosure, the needle assembly includes a needle-assembly housing, a needle-movement gear positioned in the needle-assembly housing and configured to move an injection needle from a rest position and to an injection position, a gear lock having a locked position in which the needle-movement gear cannot be rotated, and an unlocked position in which the needle-movement gear can be rotated.

In various aspects of the disclosure, the needle assembly includes a transfer gear operably coupled with the needle-movement gear to cause rotation of the needle-movement gear to move the needle, and the gear lock is configured to prevent rotation of the transfer gear when in the locked position.

In various aspects of the disclosure, the gear lock includes a spring having a first position corresponding to the locked position of the gear lock, and a biased position corresponding to the unlocked position of the gear lock.

In various aspects of the disclosure, a cassette housing is configured to house the cartridge assembly and the needle assembly, the cassette housing being comprised of a tang, wherein when the needle assembly is not fully inserted in the cassette housing, the spring of the gear lock is in the first position and gear lock is in the locked position, and, when the needle assembly is fully inserted in the cassette housing, the tang of the cassette housing causes movement of the spring of the gear lock to the biased position to enable rotation of the transfer gear.

In various aspects of the disclosure, the tang is configured to exert a force on the spring to move a spring face of the spring away from a position restricting movement of the transfer gear.

In various aspects of the disclosure, the needle assembly includes a frame post on which the spring, a lock plate, and the transfer gear are movably mounted, when in the first position, the spring and the lock plate configured to cause the transfer gear to contact a housing protrusion of the needle-assembly housing and configured to prevent the transfer gear from being in an engaged position with the needle-movement gear, and, when in the biased position, an axial force is exerted on the transfer gear that causes the transfer gear to be pushed away from contact with the housing protrusion, causes the transfer gear to move to the engaged position with the needle-movement gear, and causes the transfer gear to move the lock plate away from a position to restrict movement of the transfer gear.

In various aspects of the disclosure, the transfer gear includes a handle configured to engage with a rotation driver and to be rotated by the rotation driver.

In various aspects of the disclosure, the needle-movement gear is one of a plurality of needle-movement gears positioned in the needle-assembly housing, each of the needle-movement gears being configured to move a corresponding injection needle to and from a standby position and an injection position, the needle assembly includes a selection gear (204) configured to activate a selected needle-movement gear, wherein, in response to movement of the selected needle-movement gear to an active position proximate the transfer gear, the transfer gear is configured to rotate the selected needle-movement gear to move the injection needle corresponding to the selected needle-movement gear.

In various aspects of the disclosure, the gear lock is configured to prevent movement of the selection gear when the gear lock is in the locked position.

In various aspects of the disclosure, a cassette housing is configured to house the cartridge assembly and the needle assembly, wherein the cassette housing includes a compartment configured to house the needle assembly, a latch configured to engage with a latch receiver on the needle assembly to retain the needle assembly in the compartment, and a latch-release actuator configured to prevent the latch from being disengaged from the latch receiver when a contact surface of the needle assembly is positioned against an injection surface.

In various aspects of the disclosure, the latch-release actuator includes an actuation surface configured to be positioned against the injection surface when the contact surface of the needle assembly is positioned against the injection surface.

In various aspects of the disclosure, the latch-release actuator is configured to release the latch from being engaged with the latch receiver when a force applied to the actuation surface includes lateral component directed parallel to the contact surface of the needle assembly.

In various aspects of the disclosure, the compartment of the cassette housing includes a first opening configured to enable a first driver gear to drive a needle-selection gear of the needle assembly, and a second opening configured to enable a second driver gear to drive a movement gear of the needle assembly.

In various aspects of the disclosure, the movement gear is a transfer gear configured to cause rotation of a needle-movement gear of the needle assembly to move a needle to and from a retracted position and an injection position.

In various aspects of the disclosure, a cassette housing is configured to house a dispenser comprised of the cartridge assembly and the needle assembly, wherein the cassette housing includes a first opening configured to enable the dispenser to be inserted axially into a cavity of the cassette housing, the cavity being comprised of a first compartment configured to house the needle assembly and a second compartment configured to house the cartridge assembly, and a second opening configured to enable a motor to rotate the driver assembly.

In various aspects of the disclosure, the cassette housing includes a driver gear configured to engage with the driver assembly, and the driver gear is configured to transfer a rotational force from the motor to the driver assembly.

In various aspects of the disclosure, the dispenser is disposable and is removable from the cassette housing, and the cassette housing is reusable with another dispenser.

In various aspects of the disclosure, the cassette housing is configured to mount to a motor assembly comprised of at least one motor configured to drive movement in one or both of: the drive assembly and the needle assembly.

In various aspects of the disclosure, the cassette housing has a generally rectangular shape.

In various aspects of the disclosure, the fluid includes a medication and the cartridge includes a fluid chamber with the medication therein.

In various aspects of the disclosure, the activating of the needle of assembly includes causing a gear lock of the needle assembly to change from a locked state to an unlocked state by a protrusion of the cassette housing exerting a force on the gear lock when the dispenser is fully inserted in the cassette housing.

In various aspects of the disclosure, the force is exerted on a spring of the gear lock.

In various aspects of the disclosure, the force exerted on the gear lock enables a transfer gear to rotate a needle-movement gear.

In various aspects of the disclosure, the force exerted on the gear lock enables the needle-selector gear to rotate the needle selector.

In various aspects of the disclosure, reusing the cassette housing with a new dispenser by manipulating an actuator of the latch mechanism of the cassette to cause the latch mechanism to de-latch from the dispenser, removing the dispenser from the cassette housing, and activating the new dispenser by fully inserting the new dispenser in the cassette housing and causing the latch mechanism of the cassette housing to latch to the new dispenser to prevent movement of a needle assembly of the new dispenser relative to the cassette housing, wherein the manipulating of the latch mechanism cannot be performed while a contact surface of the cassette housing is in contact with an injection surface, the contact surface of the cassette housing being coplanar with and encircling a contact surface of the needle assembly of the dispenser.

Use of ordinal terms such as “first,” “second,” “third,” etc., in the description and the claims to modify an element does not by itself connote any priority, precedence, or order of one element over another, or the temporal order in which acts of a method are performed, but are used merely as labels to distinguish one element or act having a certain name from another element or act having a same name (but for use of the ordinal term) to distinguish the elements or acts.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein in the specification and in the claims, the phrase “equal” or “the same” in reference to two values (e.g., distances, widths, etc.) means that two values are the same within manufacturing tolerances. Thus, two values being equal, or the same, may mean that the two values are different from one another by ±5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

Also, the phraseology and terminology used herein is for the purpose of description and should not be regarded as limiting. Use of terms such as “including,” “comprising,” “comprised of,” “having,” “containing,” and “involving,” and variations thereof herein, is meant to encompass the items listed thereafter and equivalents thereof as well as additional items.

The terms “approximately” and “about” if used herein may be construed to mean within ±20% of a target value in some embodiments, within ±10 % of a target value in some embodiments, within ±5% of a target value in some embodiments, and within ±2% of a target value in some embodiments. The terms “approximately” and “about” may equal the target value.

The term “substantially” if used herein may be construed to mean within 95% of a target value in some embodiments, within 98% of a target value in some embodiments, within 99% of a target value in some embodiments, and within 99.5% of a target value in some embodiments. In some embodiments, the term “substantially” may equal 100% of the target value.

1. A fluid delivery cassette comprising:
a cartridge assembly comprising:
a cartridge configured to contain a fluid,
a septum positioned at a distal opening of the cartridge,
a movable piston sealing a proximal opening of the cartridge, the piston being movable in an axial direction of the cartridge, and
a drive assembly configured to move the piston in the axial direction toward the distal opening; and
a needle assembly attached to the cartridge assembly,
wherein the drive assembly includes:
an axially extendible portion having a distal end that is controllably extendible from a retracted position to at least one extended position along a longitudinal axis, wherein the axially extendible portion is manufactured as a tapered helix having a diameter that varies from a first end of the tapered helix to a second end of the tapered helix so that the axially extendible portion is collapsible into the retracted position, and
a rotatable force-transfer portion configured to receive an applied force and to cause the distal end of the axially extendible portion to move in the axial direction in response to the applied force.

2. The fluid delivery cassette of claim 1, wherein the force-transfer portion has a tapered profile and comprises at least one drive rib extending radially from a surface of the force-transfer portion.

3. The fluid delivery cassette of claim 1, wherein the at least one drive rib, along an axial length of the drive rib, extends a radial distance (d) from a radially outer edge of the drive rib to the longitudinal axis.

4. The fluid delivery cassette of claim 1, further comprising:
a cartridge housing comprising:
a first section configured to contain a least a portion of the axially extendible portion,
a second section configured to contain at least a portion of the cartridge, and
a third section configured to align the axially extendible portion and the cartridge.

5. The fluid delivery cassette of claim 4, further comprising:
a foot attached to the distal end of the axially extendible portion, wherein:
the foot is configured to move in the second section of the cartridge housing,
the foot comprises a foot surface configured to come into contact with a piston surface of the piston to move the piston toward the distal opening of the cartridge, and
the foot surface comprises a relief structure configured to engage with at least one corresponding surface structure on the piston surface.

6. The fluid delivery cassette of claim 1, wherein the force-transfer portion comprises:
a rotatable spindle engaged with an internal loop of a coil formed of the axially extendible portion, and
a spline portion configured to receive a spline-shaped force applicator.

7. The fluid delivery cassette of claim 6, wherein the spline portion comprises a spline-shaped recess configured to receive a spline shaft, and the spline-shaped recess and the rotatable spindle are in a coaxial relationship along the longitudinal axis.

8. The fluid delivery cassette of claim 6, wherein:
the axially extendible portion comprises a ribbon, a retracted portion the ribbon is configured to form the coil, the ribbon is configured to uncoil and extend controllably in response to the applied force, to form a helix having a controllable axial length, and the piston and the helix are coaxial, wherein
the force-transfer portion comprises a plurality of drive ribs extending radially from the surface of the force-transfer portion,
the ribbon comprises a plurality of rib latches protruding from a first surface of the ribbon, and
the rib latches are configured to slidably engage along the drive ribs when the ribbon is extending from the retracted position to the at least one extended position.

9. The fluid delivery cassette of claim 8, wherein:
an internal surface of the helix comprises the first surface of the ribbon,
a second surface of the ribbon comprises a groove configured to movably engage with and slide along a bump portion of the cartridge housing, and
in response to movement of the ribbon along the bump portion of the cartridge housing and along the drive ribs, the helix is formed from the ribbon to be columnar.

10. The fluid delivery cassette of claim 8, wherein:
the ribbon is formed of a resilient material such that, when not confined in the cartridge housing and in an absence of a force exerted on the ribbon, the ribbon forms a tapered helix having a diameter that varies from a first end of the tapered helix to a second end of the tapered helix, and
the tapered helix collapses to form a coil when an axial force is exerted on the first end or the second end or the first and second ends of the tapered helix, the axial force being a force along an axis of the tapered helix.

11. The fluid delivery cassette of claim 1, wherein:
the needle assembly is attached to a distal end of the cartridge housing, and
the septum is positioned within a recess of the needle assembly.

12. The fluid delivery cassette of claim 11, wherein the needle assembly comprises:
a needle-assembly housing,
a needle-movement gear positioned in the needle-assembly housing and configured to move an injection needle from a rest position and to an injection position,
a gear lock having:
a locked position in which the needle-movement gear cannot be rotated, and
an unlocked position in which the needle-movement gear can be rotated.

13. The fluid delivery cassette of claim 12, wherein:
the needle assembly comprises a transfer gear operably coupled with the needle-movement gear to cause rotation of the needle-movement gear to move the needle, and
the gear lock is configured to prevent rotation of the transfer gear when in the locked position, the gear lock having a spring bias.

14. The fluid delivery cassette of claim 13, further comprising:
a cassette housing configured to house the cartridge assembly and the needle assembly, the cassette housing being comprised of a tang, the tang having a spring bias,
wherein:
when the needle assembly is not fully inserted in the cassette housing, the spring of the gear lock is in a first position and the gear lock is in the locked position, and,
when the needle assembly is fully inserted in the cassette housing, the tang of the cassette housing causes movement of the spring of the gear lock to a biased position to enable rotation of the transfer gear.

15. The fluid delivery cassette of claim 14, wherein:
the needle assembly comprises a frame post on which the spring, a lock plate, and the transfer gear are movably mounted,
when in the first position, the spring and the lock plate configured to cause the transfer gear to contact a housing protrusion of the needle-assembly housing and configured to prevent the transfer gear from being in an engaged position with the needle-movement gear, and,
when in the biased position, an axial force is exerted on the transfer gear that:
causes the transfer gear to be pushed away from contact with the housing protrusion,
causes the transfer gear to move to the engaged position with the needle-movement gear, and
causes the transfer gear to move the lock plate away from a position to restrict movement of the transfer gear

16. The fluid delivery cassette of 15, wherein:
the transfer gear comprises a handle configured to engage with a rotation driver and to be rotated by the rotation driver.

17. The fluid delivery cassette of claim 12, wherein:
the needle-movement gear is one of a plurality of needle-movement gears positioned in the needle-assembly housing, each of the needle-movement gears being configured to move a corresponding injection needle to and from a standby position and an injection position,
the needle assembly comprises a selection gear configured to activate a selected needle-movement gear, wherein, in response to movement of the selected needle-movement gear to an active position proximate the transfer gear, the transfer gear is configured to rotate the selected needle-movement gear to move the injection needle corresponding to the selected needle-movement gear.

18. The fluid delivery cassette of claim 1, further comprising:
a cassette housing configured to house the cartridge assembly and the needle assembly,
wherein the cassette housing comprises:
a compartment configured to house the needle assembly,
a latch configured to engage with a latch receiver on the needle assembly to retain the needle assembly in the compartment, and
a latch-release actuator configured to prevent the latch from being disengaged from the latch receiver when a contact surface of the needle assembly is positioned against an injection surface.

19. The fluid delivery cassette of claim 18, wherein the compartment of the cassette housing comprises:
a first opening configured to enable a first driver gear to drive a needle-selection gear of the needle assembly, and
a second opening configured to enable a second driver gear to drive a movement gear of the needle assembly.

20. The fluid delivery cassette of claim 19, wherein the movement gear is a transfer gear configured to cause rotation of a needle-movement gear of the needle assembly to move a needle to and from a retracted position and an injection position.

21. The fluid delivery cassette of claim 1, further comprising:
a cassette housing configured to house a dispenser comprised of the cartridge assembly and the needle assembly,
wherein the cassette housing comprises:
a first opening configured to enable the dispenser to be inserted axially into a cavity of the cassette housing, the cavity being comprised of a first compartment configured to house the needle assembly and a second compartment configured to house the cartridge assembly, and
a second opening configured to enable a motor to rotate the driver assembly.

22. The fluid delivery cassette of claim 1, wherein the fluid comprises a medication and the cartridge includes a fluid chamber with the medication therein.

23. A fluid delivery system comprising:
the fluid delivery cassette of claim 1, and
a reusable cassette housing configured to contain at least a portion of the fluid delivery cassette; and
a reusable motor assembly configured to drive movement in the cartridge assembly or in the needle assembly or in both the cartridge assembly and the needle assembly,
wherein the motor assembly is removably mountable on the cassette housing.
